(19) United States
(12) Patent Application Publication
(10) Pub. No.: US 2016/0083722 A1
(43) Pub. Date: Mar. 24, 2016

(54) COMPOSABILITY AND DESIGN OF PARTS FOR LARGE-SCALE PATHWAY ENGINEERING IN YEAST

(71) Applicant: Massachusetts Institute of Technology, Cambridge, MA (US)

(72) Inventors: Eric M. Young, Arlington, MA (US); David Benjamin Gordon, Somerville, MA (US); Christopher Voigt, Belmont, MA (US)

(73) Assignee: Massachusetts Institute of Technology, Cambridge, MA (US)

(21) Appl. No.: 14/838,409

(22) Filed: Aug. 28, 2015

Related U.S. Application Data
(60) Provisional application No. 62/043,466, filed on Aug. 29, 2014.

(51) Int. Cl. C12N 15/10; C12N 15/113

(52) U.S. Cl. CPC C12N 15/1082; C12N 15/113

(57) ABSTRACT

Expression cassettes comprising promoter and terminator combinations are provided and can be used to tune gene expression. Synthetic yeast promoters and methods of making them also are provided.

### RELATED APPLICATION

This application claims the benefit under 35 U.S.C. §119(e) of U.S. provisional application 62/043,466, filed Aug. 29, 2014, the entire disclosure of which is incorporated herein by reference.

### FIELD OF INVENTION

Composability of yeast promoters and terminators are provided in the construction of libraries of expression cassettes to control gene expression and design of synthetic yeast promoters are provided that may be incorporated into the expression cassettes.

### BACKGROUND OF INVENTION

A central goal of synthetic biology is achieving precise control of gene expression [1]. In pursuit of this goal, a variety of tools have been developed to tune gene expression at the levels of transcription and translation in the yeast Saccharomyces cerevisiae [1-5].

Several recent studies have developed either promoter libraries or terminator libraries [5-7]. These transcriptional part libraries have been shown to enable graded expression across wide ranges. While this finding was anticipated for promoters, it is rather unexpected that a yeast terminator not only stops transcription, but has expression-enhancing properties (likely due to determining the degree of polyadenylation and thus half-life of the resultant mRNA) [8].

With these findings, it becomes necessary to consider interactions when these parts are used in conjunction to tune gene expression; in other words, the composability of promoters and terminators. Recent work has shown that composability is a concern when designing transcriptional units in E. coli [9], therefore it is reasonable to consider that yeast transcriptional parts will interact in (as yet) unpredictable ways. Therefore, a paradigm shift of gene expression in yeasts and perhaps all eukaryotes must take place: the promoter and terminator must be treated as an expression cassette with a corresponding expression strength value.

No study that varies only one part type can investigate expression cassettes and part composability; as a result, it was, until this study, impossible to predict the gene expression strength of a new promoter-terminator combination.

Furthermore, existing part libraries are not redundant, that is, they define only one particular part at a given expression strength. In practice, a given expression strength may be required more than once in a genetic design. However, current parts libraries would require the reuse of a part to achieve the same level of expression. This invites instability due to the active homologous recombination machinery in Saccharomyces cerevisiae. If multiple part combinations produced the same expression cassettes, these would be very useful in the art of gene expression balancing.

Recent work in the field has begun to unravel the sequence features of yeast promoters, and how the degree of transcriptional activation depends on these features. The two primary sequence features of yeast promoters are binding sites for transcription factors and varying nucleotide percentages at specific regions in the promoter. Transcription factors are thought to have a dual role of disrupting DNA-sequestering nucleosomes while binding with elements of the transcription initiation complex [13, 14]. Changing nucleotide content is also thought to create nucleosome-free regions, and, in the 5′-UTR, influence translation rates of the resultant mRNA [15]. Notably, it has been shown that specific nucleotide content patterns in the core promoter correlate with promoter expression strength [15].

Furthermore, it has been shown that synthetic promoters may be created by seemingly arbitrary arrangements and combinations of transcription factors, or by random sequences projected to have low nucleosome occupancy [12, 13]. However, transcription factor shuffling experiments were not designed with any predetermined idea of strength nor are these promoters easily used in large-scale assembly of genetic designs because of a high degree of homology. Similarly, designing promoters based on nucleosome occupancy is computationally expensive and therefore low-throughput.

### SUMMARY OF INVENTION

An expression cassette (promoter-terminator) library is needed for which expression strength is known and predictable and that has expression cassette redundancy (different parts, same strength). This will enable addition of thousands of new parts for which transcriptional strength is known and predictable. In addition, a method of designing fully synthetic yeast promoters according to desired strength was devised. This is an advance beyond random methods recently published [12].

According to one aspect, libraries of expression cassettes are provided. The libraries include a plurality of expression cassettes, each comprising a promoter and a terminator; wherein each of the promoters and terminators is different from all of the other promoters and terminators in the plurality of expression cassettes; and wherein each of the promoters and terminators or each combination of a promoter and a terminator has a known or predicted expression strength. In some embodiments, the promoter and the terminator flank an insertion site for a nucleic acid molecule to be expressed. In some embodiments, each expression cassette of at least a first subset of the plurality of expression cassettes has about the same expression strength. In some embodiments, each expression cassette of a second subset of the plurality of expression cassettes has about the same expression strength, which expression strength is different than the expression strength of the first subset of the plurality of expression cassettes.

In some embodiments, one or more of the promoters are constitutive promoters. In some embodiments, one or more of the promoters are synthetic promoters. In some embodiments, one or more of the terminators are expression-enhancing terminators. In some embodiments, one or more of the terminators are synthetic terminators. In some embodiments, there is less than 40 bp contiguous identity between promoter sequences to prevent recombination. In some embodiments, there is less than 40 base pairs (bp) contiguous identity between terminator sequences.

In some embodiments, the expression cassettes are comprised within a plurality of plasmids. In some embodiments, the plurality of expression cassettes or the plurality of plasmids is at least 5 different expression cassettes or at least 5 different plasmids.

In some embodiments, the expression cassettes or plasmids are assembled using Type IIS cloning. In some embodiments, the expression cassette flanked by sequences with sufficient identity to yeast chromosome sequences to permit integration of the expression cassette into the yeast genome.

According to another aspect, methods of making a library of expression cassettes are provided. The methods include selecting promoter and terminator sequences for assembly into the expression cassettes by (1) limiting identity among and between sequences to less than 40 bp contiguous identity; (2) varying promoter strengths determined by transcriptomics and expression data; (3) including homologs to strong S. cerevisiae promoters from other yeasts; (4) using expression-enhancing terminators; (5) using only promoter and terminator sequences from constitutive genes; and/or (6) using promoter and terminator sequences that have no genome annotation describing known regulatory elements, ORFs, or centromeres; assembling the selected promoter and terminator sequences into the expression cassettes; and measuring the expression strength of the expression cassettes or predicting the expression strength of the expression cassettes via a model. In some embodiments, the model is an empirical model that predicts the expression of any promoter-terminator combination.

In some embodiments, the assembling the selected promoter and terminator sequences into the expression cassettes is performed by: providing a plurality of promoter sequences, a plurality of terminator sequences, and a selection cassette sequence, wherein: the promoter sequences are flanked 5′ by a sequence that has identity with a sequence that is 5′ to an integration site on a yeast genome, and are flanked 3′ by a fragment of a detectable marker; the terminator sequences are flanked 5′ by an overlapping fragment of the detectable marker, wherein the two fragments of the detectable marker comprise sufficient sequence when combined to express a functional detectable marker, and are flanked 3′ by a sequence that has identity with a selection cassette sequence; and the selection cassette sequence is flanked 5′ by a sequence that has identity with a sequence that is 3′ to the terminator sequences, and is flanked 3′ by a sequence that has identity with a sequence that is 3′ to an integration site on a yeast genome, combining the promoter sequences, the terminator sequences and the selection cassette sequence to prepare different combinations of promoter sequences and terminator sequences with the selection cassette sequence, transforming the combinations of sequences into yeast cells, and recombining and integrating the combinations of sequences into the genome of the yeast cells via homologous recombination.

In some embodiments, the promoter, terminator, and selection cassette sequences are PCR-amplified sequences. In some embodiments, the detectable marker is a sequence encoding a fluorescent protein. In some embodiments, the selection cassette is an auxotrophic selection cassette or an antibiotic selection cassette. In some embodiments, the auxotrophic selection cassette is a HIS selection cassette, a LEU selection cassette, a URA selection cassette, a TRP selection cassette, a LYS selection cassette, or a MET selection cassette. In some embodiments, the antibiotic selection cassette is a KanMX selection cassette, a NatMX selection cassette, an hphMX6 selection cassette or a bleMX6 selection cassette.

In some embodiments, the promoter sequences, the terminator sequences, and the selection cassette sequence are combined using a robotic or programmed liquid handler. In some embodiments, the methods also include testing the expression of the detectable marker in the yeast cells to determine the expression strength of the combinations of the promoter and terminator sequences.

According to another aspect, methods for constructing a genetic design are provided. The methods include selecting a plurality of expression cassettes from the foregoing libraries and cloning an open reading frame sequence of the genetic design between the promoter and terminator sequences of each of the plurality of expression cassettes. In some embodiments, the plurality of expression cassettes is selected based on measuring the expression strength of the expression cassettes or predicting the expression strength of the expression cassettes via a model. In some embodiments, the model is an empirical model that predicts the expression of any promoter-terminator combination. In some embodiments, the genetic design is a genetic pathway or circuit. In some embodiments, the genetic pathway or circuit is a metabolic pathway or a synthetic gene circuit.

In some embodiments, the cloning includes assembling the promoter sequences, open reading frame sequences, and terminator sequences in a yeast cell by homologous recombination. In some embodiments, the promoter sequences are flanked 5′ by a sequence that has identity with a sequence that is 5′ to an integration site on a yeast genome, and are flanked 3′ by a fragment of an open reading frame sequence; the terminator sequences are flanked 5′ by an overlapping fragment of the open reading frame sequence, wherein the two fragments of the open reading frame sequence comprise sufficient sequence when combined to express a functional open reading frame sequence, and are flanked 3′ by a sequence that has identity with a selection cassette sequence; and the selection cassette sequence is flanked 5′ by a sequence that has identity with a sequence that is 3′ to the terminator sequences, and is flanked 3′ by a sequence that has identity with a sequence that is 3′ to an integration site on a yeast genome.

In some embodiments, the assembling includes: transforming the promoter sequences, open reading frame sequences, and terminator sequences into yeast cells, and recombining and integrating the promoter sequences, open reading frame sequences, and terminator sequences into the genome of the yeast cells via homologous recombination. In some embodiments, the methods also include expressing the genetic pathway or circuit.

According to another aspect, synthetic promoters comprising nucleotide sequences of anticipated strength and promoter element sequences are provided. In some embodiments, the nucleotide sequences of anticipated strength have nucleotide content that correlates with a predetermined expression strength, the promoter element sequences are selected for probable expression strength, and the nucleotide sequences of anticipated strength are interspersed with the promoter element sequences.

In some embodiments, the nucleotide sequences of anticipated strength and promoter element sequences do not comprise Type IIS restriction endonuclease recognition sequences, ATG sequences, or sequences that bind non-coding RNA degradation proteins NAB3 and NRD1. In some embodiments, the nucleotide sequences of anticipated strength are sequences that have nucleotide content patterns consistent with expected expression strengths.

According to another aspect, methods of preparing synthetic yeast promoters are provided. The methods include generating nucleotide sequences of an upstream activation sequence 2 (UAS2), an upstream activation sequence 1 (UAS1), and a core comprising a TATA binding protein (TBP) region, a transcription start site (TSS), and a 5′ untranslated region (UTR), wherein the nucleotide sequences satisfy constraints on the nucleotide sequences and are generated based on a predetermined expression strength and promoter element types that are included in the UAS2, UAS1, and core; substituting promoter element sequences at predetermined locations in the UAS2, UAS1, and core; and optionally synthesizing the nucleotide sequences.

In some embodiments, the nucleotide sequences have nucleotide content patterns consistent with expected expression strengths. In some embodiments, the promoter element sequences substituted at specific locations are selected from the group consisting of transcription factor binding site sequences, poly A/T sequences, TATA box sequences, transcription start element sequences, and Kozak element sequences. In some embodiments, the steps of generating nucleotide sequences and substituting promoter element sequences comprise synthesizing oligonucleotides comprising portions of the nucleotide sequences. In some embodiments, the methods also include removing Type IIS restriction endonuclease recognition sequences, ATG sequences and sequences that bind non-coding RNA degradation proteins NAB3 and NRD1 from the nucleotide sequences and the promoter element sequences prior to synthesizing the nucleotide sequences.

According to another aspect, methods of preparing synthetic yeast promoters are provided. The methods include generating nucleotide sequences of an upstream activation sequence 2 (UAS2), an upstream activation sequence 1 (UAS1), or a core comprising a TATA binding protein (TBP) region, a transcription start site (TSS), and a 5′ untranslated region (UTR), wherein the nucleotide sequences are generated based on a predetermined expression strength and promoter element types that are included in the UAS2, UAS1, or core; substituting promoter element sequences at predetermined locations in the UAS2, UAS1, or core to produce a synthetic UAS2 sequence, UAS1 sequence, or core sequence; synthesizing the nucleotide sequences; and replacing a part of a yeast promoter with one or more of the synthetic UAS2 sequence, the UAS1 sequence, and the core sequence.

In some embodiments, the nucleotide sequences have nucleotide content patterns consistent with expected expression strengths. In some embodiments, the methods also include removing Type IIS restriction endonuclease recognition sequences, ATG sequences, and sequences that bind non-coding RNA degradation proteins NAB3 and NRD1 from the random sequences and the promoter element sequences prior to synthesizing the nucleotide sequences. In some embodiments, the synthetic UAS2 sequence, UAS1 sequence, or core sequence are a plurality of synthetic sequences and wherein replacing the part of the yeast promoter with one or more of the plurality of synthetic UAS2 sequences, the plurality of UAS1 sequences, and the plurality of core sequences produces a library of synthetic yeast promoters having one or more of the UAS2, UAS1, and core sequences replaced. In some embodiments, the methods also include cloning a nucleotide sequence that encodes a detectable marker downstream of the synthetic yeast promoter(s). In some embodiments, the methods also include expressing the detectable marker and measuring the expression strength of the synthetic yeast promoter(s). In some embodiments, the detectable marker is a sequence encoding a fluorescent protein.

In some embodiments, the yeast promoter of which a part is replaced with one or more of the synthetic UAS2 sequence, the UAS1 sequence, and the core sequence is a TEF1 promoter, a TDH3 promoter, or a variant based on the TDH3 promoter.

### BRIEF DESCRIPTION OF THE DRAWINGS

The accompanying drawings are not intended to be drawn to scale. For purposes of clarity, not every component may be labeled in every drawing.

FIG. 1A. Summary of part types and selection strategies.

FIG. 1B. Summary of hybrid Type IIS “GoldenGate” and homologous recombination method for parts characterization. Building characterization cassettes using the PCR fragment method shown, which requires correct recombination of a partial GFP gene and a NatMX selection, has not been previously demonstrated.

FIGS. 2A-2D. Expression strengths of integrated promoter-terminator cassettes in S.c. CENPK-113.

FIG. 2A. Heatmap of GFP expression resulting from promoter-terminator combinations. Four orders of magnitude of expression are possible.

FIG. 2B. Model predicting bulk behavior of a given part and the comparison of model predicted values vs. measured GFP expression. Model fits well to the data.

FIG. 2C. Predicted vs. measured GFP expression with P2 and P7 highlighted. A bar chart is shown comparing P2 and P7.

FIG. 2D. Comparison of P2 and P7. This chart shows different expression strengths between the two promoters across all terminators.

FIG. 3A. Enlarged view of FIG. 3A, Glucose, with part names instead of numbers.

FIG. 3B. Enlarged view of FIG. 3A, Galactose, with part names instead of numbers.

FIG. 4A. Expanded part set with inducible promoters GAL1p (P37) and CUP1p (P38) & DSM promoters (P39-P44) and terminators (T37-T39), Glucose.

FIG. 4B. Expanded part set with inducible promoters GAL1p (P37) and CUP1p (P38) & DSM promoters (P39-P44) and terminators (T37-T39), Galactose. Note activation of GAL1p (P37) under these conditions. P35 also appears activated.

FIG. 5A. Part context effects with efficient termination, it does not appear that transcription units are subject to read-through, although a more extensive experiment demonstrating this is forthcoming.

FIG. 5B. Part context effects correlation between transcription units expressing GFP or BFP. There is significant correlation, indicating that expression strengths are robust to different mRNA sequences, although severe mRNA secondary structure may cause ORF-specific context effects.

FIG. 6A. Replicate library that spans three orders of magnitude, accounting for promoter and terminator composability.

FIG. 6B. These expression units with known and predicted strengths may now be used to construct large combinatorial libraries of genetic designs with specific expression requirements. Brief description of a pathway assembly strategy using promoter-terminator combinations to tune gene expression. Simple diagram of the hierarchical pathway assembly strategy enabled by Type IIS cloning.

FIGS. 7A-7B. Brief description of a pathway assembly strategy using promoter-terminator combinations to tune gene expression.

FIG. 7A. Assembly diagram of the hierarchical pathway assembly strategy enabled by Type IIS cloning of the first 96 designs.

FIG. 7B. Assembly diagram of the second 96 designs.

FIG. 8A. Definition of a promoter and sequence creation flow in the ProGenie algorithm. The promoter is divided into two upstream activating sequence segments and a core segment. Random sequence is created first and then motifs are substituted. A promoter with all possible substitutions would appear as the annotated diagram.

FIG. 8B. Visual diagram of ProGenie settings for anticipated strength, nucleotide content (pie charts), and sequence motifs (bar charts).

FIG. 9. GFP expression levels of synthetic promoters compared to ACT1p and S. cerevisiae without GFP. Promoters function in accordance with expected strength designed by ProGenie.

FIG. 10. Description of experimental approach and cloning strategy for massively parallel promoter synthesis. Thirty thousand of each promoter segment (e.g. UAS2, UAS1, and core) are cloned into the yeast TEF1 promoter and then integrated into the yeast genome. Cell sorting can then select populations of cells with different levels of GFP expression. Sequencing these populations can then reveal which segments enhance the strength of expression.

FIGS. 11A-11B. Library diversity and composition before sorting.

FIG. 11A. Plots of side scatter (SSC) versus GFP fluorescence for the synthetic promoter libraries and some controls. This visually displays the diversity and range of expression strengths achieved with 30 k synthetic sequences for each of the three promoter segments. The gates drawn on the plots are rough approximates of the actual gates used to sort the libraries. After plating, picking individual colonies, confirming activity via flow cytometry, and sequencing unique clones, 16 different unique sequences have been identified to date.

FIG. 11B. Expression strength of each of the verified unique synthetic sequences.

FIG. 12. Comparison of initial synthetic promoters with three standard terminators and reference promoters. Promoters span the medium range of activity and generally fall in the order of strength in which they were designed.

### DETAILED DESCRIPTION OF DISCLOSURE

The requirements for known expression strength, composability, and redundancy necessitate a large library of parts and a system for using and adding new parts. Therefore, new characterization methods must be devised to characterize hundreds of parts and part combinations. Furthermore, models and standards must be developed to enable ease of use and expansion of the parts library. Like next-generation parts libraries that already exist [10], the assembly standard chosen for this library is based on Type IIS assembly methods [11].

By incorporating all of these considerations of strength, composability, redundancy, characterization, and standardization, the S. cerevisiae parts libraries and methods disclosed herein significantly advance the state-of-the-art.

Using a novel method to construct expression libraries has direct relevance for pathway engineering and synthetic biology, while the findings raise fundamental questions of transcription and translation control in yeast. Using the disclosed approaches one can create new parts libraries characterized in context of promoter-terminator interactions; utilize redundant parts that have the same expression strength but different sequence; utilize a large-scale part characterization method to model parts function; and utilize this model to predict new part behavior using a small number of measurements. With knowledge of transcriptional part behavior on a large scale, pathways may be optimized with confidence in anticipated expression strengths. Hypotheses can also begin to be formed as to what interactions cause the small (˜±10%) deviations from the model. It may be that transcriptional looping of genomic DNA causes promoters and terminators to come into close proximity and therefore interact. It may also be that looping of the mRNA during translation is the cause of the interaction. Whatever these effects, they seem to be only a minor component contributing to the measured expression strength, since a simple second order model that does not account for these types of interactions fits the data extremely well.

Combining the promoter and terminator as a unique expression cassette can be a powerful tool to reliably control gene expression in yeast. By using a large number of parts, redundant expression levels may be achieved using different combinations of parts. Genetic designs that require equal expression of two different genes are more stable because parts are not repeated to achieve the same strength. Implementing assembly standards allows ease of cloning and flexibility to a wide range of genetic designs. By incorporating these three qualities (treating the promoter-terminator as a cassette, expression redundancy, and standardization) into one expression library, this work represents a significant advance over the state-of-the-art.

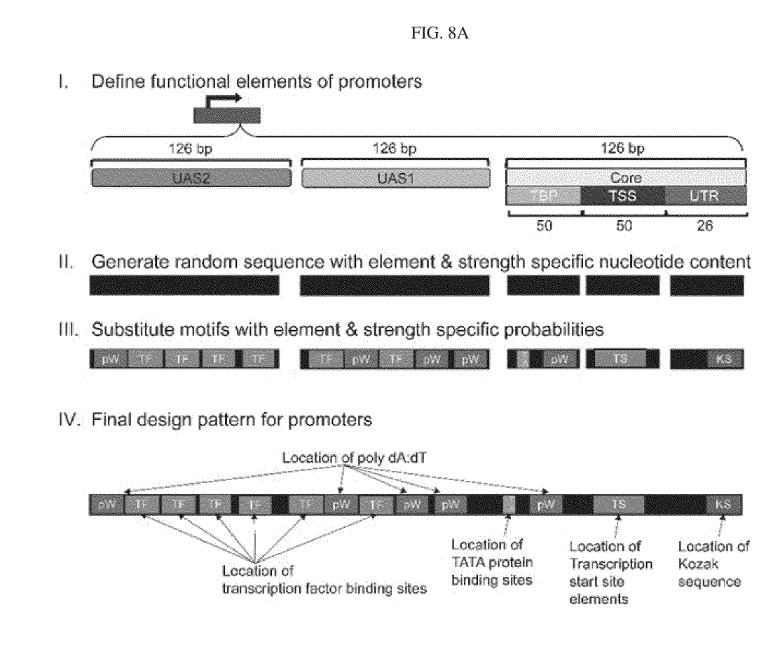

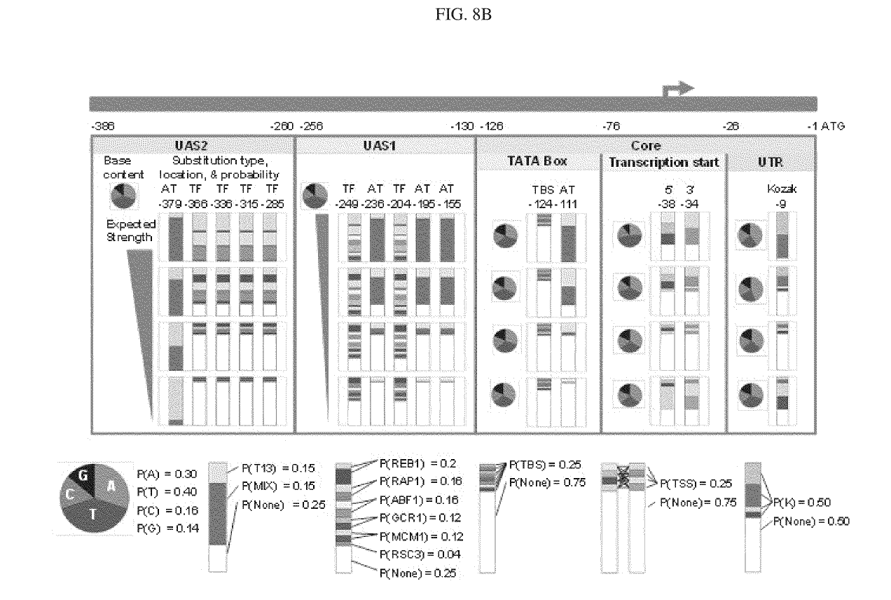

For large-scale synthetic promoter design, all known strength-enhancing binding sites and sequence features were combined into one high-throughput synthesis strategy, with sequence generation performed by a greedy constraint-based algorithm (ProGenie) for designing yeast promoters implemented in Python. This algorithm uses constraints on nucleotide content to design synthetic sequences, and then a further set of constraints to substitute various strength-enhancing sequence motifs, as shown in FIG. 8A. The algorithm is not computationally expensive, unlike design strategies based on nucleosome occupancy, and can thus design tens of thousands of promoter sequences in a matter of minutes.

The constraints on nucleotide content and motif substitution probability also change with the concept of “anticipated strength”. This is to produce a variety of different strength synthetic promoters. This is implemented as a set of four strength tiers in the algorithm, and the constraints on the sequence design are unique to each tier. Generally, motif substitution probability increases with increasing strength, graphically displayed in FIG. 8B.

The algorithm also incorporates a sequence editing functionality that removes undesired sequences that arise randomly and from substitution. There are three types of ‘undesired’ sequences in the algorithm. First are Type IIS sites that are used in subsequent cloning steps. Second are upstream ATG sites that may arise in the promoter near the start of the gene. It has been shown that upstream ATG sites dramatically decrease translational efficiency. Third are sequences that bind non-coding RNA degradation proteins NAB3 and NRD1. As many yeast promoters are naturally bidirectional, these signals exist as a way to rapidly degrade transcription initiated in the non-coding direction. However, if they arose in the synthetic sequences, it is likely that they would reduce the half-life of the resultant mRNAs, ultimately reducing the expression strength of the promoter.

Libraries of promoter and terminator combinations and methods to make expression cassettes containing them are described herein for use in tuning gene expression. Also described herein, are methods to design and make synthetic yeast promoters and their incorporation into the expression cassettes.

In some embodiments, libraries of expression cassettes are designed with promoter and terminator combinations. An expression cassette may refer to a construct of genetic material that contains coding sequences and enough regulatory information to direct proper transcription and translation of the coding sequences in a recipient cell. The expression cassette can be part of a nucleic acid vector used for cloning and transformation and targeting into a desired host cell and/or subject. With each successful transformation, the expression cassette directs a cell's machinery to make RNA and, depending on the nature of the transcribed RNA, protein. Some expression cassettes are designed for modular cloning of protein-encoding sequences so that the same cassette can easily be altered to make different proteins [34].

An expression cassette is composed of sequences controlling the expression of one or more genes or other nucleic acid sequences. Although the expression cassettes exemplified herein are designed for use in yeast, different expression cassettes can be transformed into different organisms including yeast, bacteria, plants, and mammalian cells as long as the correct regulatory sequences are used. An expression cassette includes at least a promoter sequence and a terminator sequence. In some embodiments, an expression cassette contains a promoter and a terminator. In other embodiments, an expression cassette contains a promoter and a terminator flanking an insertion site for a nucleic acid sequence. In other embodiments, an expression cassette comprises a promoter and a terminator flanking a nucleic acid molecule coding for an RNA or protein of interest. Expression cassettes also may include a 3′ untranslated region that, in eukaryotes, usually contains a polyadenylation site, one or more sequences coding for a selectable marker, and/or other sequences of interest as are known to one of skill in the art.

A promoter is a nucleotide sequence to which RNA polymerase binds to begin transcription. The promoter is required for correct transcription initiation. The promoter nucleotide sequence is capable of controlling the expression of a coding sequence or functional RNA. In general, a coding sequence is located 3′ to a promoter sequence. The promoter sequence consists of proximal and more distal upstream elements, the latter elements often referred to as enhancers. Accordingly, an enhancer is a nucleotide sequence that can stimulate promoter activity and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic nucleotide segments. It is understood by those skilled in the art that different promoters may direct the expression of a gene in different tissues or cell types, or at different stages of development, or in response to different environmental conditions.

A promoter may be constitutive, synthetic, inducible, activatable, repressible, tissue-specific, or any combination thereof. A promoter may be one naturally associated with a gene or sequence, as may be obtained by isolating the 5′ non-coding sequences located upstream of the coding segment of a given gene or sequence. Such a promoter can be referred to as “endogenous.”

A promoter may contain sub-regions at which regulatory proteins and molecules may bind, such as RNA polymerase and other transcription factors. A promoter drives expression or drives transcription of the nucleic acid sequence that it regulates. Engineered expression cassettes of the present disclosure comprise, in some embodiments, promoters operably linked to a nucleotide sequence (e.g., encoding a protein of interest). A promoter is considered to be operably linked when it is in a correct functional location and orientation in relation to the nucleotide sequence that it regulates, to control (drive) transcriptional initiation and/or expression of that sequence. A promoter is a control region of a nucleic acid at which initiation and rate of transcription of the remainder of a nucleic acid are controlled. A promoter may be classified as strong or weak according to its affinity for RNA polymerase (and/or sigma factor); this is related to how closely the promoter sequence resembles the ideal consensus sequence for the polymerase. The strength of a promoter may depend on whether initiation of transcription occurs at that promoter with high or low frequency. Different promoters with different strengths may be used to construct nucleic acids with different levels of gene/protein expression (e.g., the level of expression initiated from a weak promoter is lower than the level of expression initiated from a strong promoter).

In some embodiments, libraries of expression cassettes are constructed, wherein the plurality of expression cassettes have about the same expression strength. In some embodiments, the combination of promoters and terminators used in the construction of the library of expression cassettes tunes expression strength. “About the same expression strength” refers to a comparison in gene expression from two or more expression cassettes in a plurality of expression cassettes, wherein the expression is the same, or wherein the difference in expression between the expression cassettes is, for example, ±1%, ±2%, ±3%, ±4%, ±5%, ±6%, ±7%, ±8%, ±9%, ±10%, ±11%, ±12%, ±13%, ±14%, ±15%, ±16%, ±17%, ±18%, ±19% or ±20%.

In other embodiments, expression cassettes of different expression strength are provided in one or more libraries. For example, there may be sets of expression cassettes of about the same expression strength that differ in expression strength from other sets of expression cassettes. Thus a library can contain two or more sets of expression cassettes that provide expression strengths that are about the same within a set, but different between the sets. In these embodiments, “different expression strength” refers to a difference of more than ±20%, ±30%, ±40%, ±50%, ±60%, ±70%, ±80%, ±90, ±100%, ±120%, ±130%, ±140%, ±150%, ±160%, ±170%, ±180%, ±190, ±200%, ±300%, ±400%, ±500%, or more.

Parts (e.g. promoters, terminators, and/or sequences within an insertion site of the expression cassette) may be used to tune gene expression according to predetermined ratios of expression that are required to attain about the same expression strength. The similarities and/or differences in expression strength of expression cassettes permit selection of expression cassettes based, for example, on the ratios of expression required.

Several known yeast promoters may be used to construct expression cassettes or expression plasmids. In some embodiments, the core sequence of the promoter in the expression cassette or of the synthetic promoter is a translational elongation factor EF-1 alpha (TEF1) promoter, a triose-phosphate dehydrogenase (TDH3) promoter, or a variant based on the TDH3 promoter. Variants of the yeast TDH3 promoter in which the TATA box element is replaced by at least another sequence containing a consensus TATA site may be used in some embodiments. In some embodiments, the TDH3 TATA box element may be replaced by a portion of the phage lambda operator containing a consensus TATA site flanked by binding sites for the cI transcriptional repressor protein. Other promoters that can be used in expression cassettes include ADH1, TPI1, HXT7, PGK, PYK1, GAL1, and GAL10.

In some embodiments, nucleotide sequence may be placed under the control of a recombinant or heterologous promoter, which refers to a promoter that is not normally associated with the nucleotide sequence in its natural environment. Such promoters may include promoters of other genes; promoters isolated from any other prokaryotic cell; and synthetic promoters that are not “naturally occurring” such as, for example, those that contain different elements of different transcriptional regulatory regions and/or mutations that alter expression, as are described elsewhere herein. In addition to producing nucleotide sequences of promoters synthetically, sequences may be produced using recombinant cloning and/or nucleic acid amplification technology, including polymerase chain reaction (PCR).

In some embodiments, the expression cassettes comprise a constitutive promoter. A constitutive promoter is unregulated and allows for continual transcription of its associated gene.

In some embodiments, the expression cassettes comprise a synthetic promoter. A synthetic promoter is a DNA sequence that does not exist in nature that has been designed to control expression of a target gene.

In some embodiments, combinations of promoters and terminators are used in the construction of the expression cassettes to tune gene expression. In some embodiments, the expression cassette comprises a terminator, which is a nucleic acid sequence that signals the end of transcription. The terminator sequence mediates transcriptional termination by providing signals in the newly synthesized mRNA that trigger processes which release the mRNA from the transcriptional complex. Those processes include the direct interaction of the mRNA secondary structure with the complex and/or the indirect activities of recruited termination factors. Release of the transcriptional complex frees RNA polymerase and related transcriptional machinery to begin the transcription of new mRNAs.

In some embodiments, the terminator is an expression-enhancing or “high-capacity” terminator. In addition to stopping transcription, expression-enhancing terminators may enhance the expression of a gene, likely due to differing degrees of polyadenylation, which may influence the half-life of the resultant mRNA [5, 8]. In some embodiments, the terminator is an expression-influencing terminator. Expression-influencing terminators may either enhance or repress expression.

A nucleic acid molecule refers to the phosphate ester form of ribonucleotides (RNA molecules) or deoxyribonucleotides (DNA molecules), or any phosphodiester analogs, in either single-stranded form, or a double-stranded helix. Double-stranded DNA-DNA, DNA-RNA and RNA-RNA helices are possible. The term nucleic acid molecule, and in particular DNA or RNA molecule, refers to the primary and secondary structure of the molecule, and does not limit it to any particular tertiary forms. Thus, this term includes double-stranded DNA found, inter alia, in linear (e.g., restriction fragments) or circular DNA molecules, plasmids, and chromosomes. In discussing the structure of particular double-stranded DNA molecules, sequences may be described according to the normal convention of giving only the sequence in the 5′ to 3′ direction along the nontranscribed strand of DNA (i.e., the strand having a sequence homologous to the mRNA).

The terms “nucleic acid” and “nucleic acid molecule,” as used interchangeably herein, refer to a compound comprising a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses single and/or double stranded RNA as well as single and/or double-stranded DNA. Nucleic acids may be naturally occurring, for example, in the context of a genome, transcript, messenger RNA (mRNA), transfer RNA (tRNA), ribosomal RNA (rRNA), small nuclear RNA (snRNA), plasmid, chromosome, chromatid, or other naturally occurring nucleic acid molecule. A nucleic acid molecule may be non-naturally occurring or artificial, e.g., a peptide nucleic acid (PNA), morpholino- and locked nucleic acid (LNA), glycol nucleic acid, threose nucleic acid, short-hairpin RNA (shRNA), small-interfering RNA (siRNA), or including non-naturally occurring nucleotides or nucleosides. Artificial nucleic acids may be distinguished from naturally occurring DNA or RNA through changes to the backbone of the molecule. Furthermore, the terms “nucleic acid,” “DNA,” “RNA,” and/or similar terms include nucleic acid analogs, i.e. analogs having other than a phosphodiester backbone.

Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

A recombinant nucleic acid molecule is a nucleic acid molecule that has undergone a molecular biological manipulation, i.e., non-naturally occurring nucleic acid molecule or genetically engineered nucleic acid molecule. Furthermore, recombinant DNA molecule refers to a nucleic acid sequence which is not naturally occurring, or can be made by the artificial combination of two otherwise separated segments of nucleic acid sequence, i.e., by ligating together pieces of DNA that are not normally continuous. An artificial combination of recombinant DNA is often produced by either chemical synthesis means, or by the artificial manipulation of isolated segments of nucleic acids, e.g., by genetic engineering techniques using restriction enzymes, ligases, and similar recombinant techniques as described by, for example, Sambrook et al., Molecular Cloning, second edition, Cold Spring Harbor Laboratory, Plainview, N.Y.; (1989), or Ausubel et al., Current Protocols in Molecular Biology, Current Protocols (1989), and DNA Cloning: A Practical Approach, Volumes I and II (ed. D. N. Glover) IREL Press, Oxford, (1985); each of which is incorporated herein by reference.

In some embodiments, a plurality of expression cassettes is constructed wherein identity of the promoters and/or identity of the terminators is/are limited as assessed by alignment and/or identity of the promoter sequences in order to prevent homologous recombination in yeast. In some embodiments, in a plurality of expression cassettes, the identity among and between the promoters and/or among and between the terminators is limited to 40 base pairs (bp) contiguous identity, wherein contiguous identity among and between the sequences may be a length of not more than 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 bp. Thus, a promoter may have high percent identity but still have low rates of recombination because the segments which are identical are not contiguous for more than 39 bp, including any length from 40 bp up to the full length of the shorter sequence. Therefore, in some embodiments, where the promoters and/or terminators are partially identical, the identity over a sequence alignment may be contiguous for less than 40 base pairs, including not more than 10, 11, 12, 13, 14, 15, 16, 17, 18, 19, 20, 21, 22, 23, 24, 25, 26, 27, 28, 29, 30, 31, 32, 33, 34, 35, 36, 37, 38, or 39 bp.

Limiting the identity of promoters and/or terminators within expression cassette libraries to less than a 40 bp contiguous sequence, as described above, may prevent homologous recombination in yeast.

The term alignment defines the process or result of matching up the nucleotide or amino acid residues of two or more biological sequences to achieve maximal levels of identity and, in the case of amino acid sequences, conservation, for the purpose of assessing the degree of similarity and the possibility of homology. The term homology refers to the similarity attributed to descent from a common ancestor. The term homologous is a term understood in the art that refers to nucleic acids or polypeptides that are highly related at the level of nucleotide or amino acid sequence. Homologous biological molecules or components (nucleic acids, genes, proteins, polypeptides, structures) are called homologs or homologues. The term identity refers to the extent to which two nucleotide or amino acid sequences have the same residues at the same positions in an alignment, often expressed as a percentage. In some embodiments, identity of promoters and terminators within a plurality of expression cassettes is limited by length of contiguous identity, as described above.

The term homologous recombination, also termed general recombination or recombination, generally refers to a process in which genetic exchange takes place between a pair of homologous DNA sequences. Homologous recombination refers to a process in which homologous and/or identical nucleic acid molecules are broken and the fragments are rejoined in new combinations. This can occur in the living cell, e.g. through crossing-over during meiosis, or in vitro i.e. during cloning processes. Homologous recombination relies on extensive base-pairing interactions between two nucleic acid sequences that recombine, occurring only between homologous DNA molecules. In the present invention, homologous recombination is prevented by limiting the contiguous identity of sequences within a plurality of expression cassettes.

The terms recombine and recombination, in the context of a nucleic acid modification (e.g., a genomic modification), may refer to the process by which two or more nucleic acid molecules, or two or more regions of a single nucleic acid molecule, are modified by the action of restriction enzymes, DNA ligases, recombinases, and/or successive hybridization assembling (SHA), a denaturation/renaturation treatment. Recombination may result in, inter alia, the insertion, inversion, excision, or translocation of a nucleic acid sequence, e.g., in or between one or more nucleic acid molecules.

In some embodiments, the amount of gene expression from a nucleic acid molecule is tuned through the use of a combination of promoters and terminators within a plurality of expression cassettes or a plurality of plasmids. Gene expression is a process by which information from a gene may be used for synthesizing a functional gene product. The functional gene product can be a protein. Non-protein coding genes, such as transfer RNA (tRNA) or small nuclear RNA (snRNA), can encode a functional RNA.

In some embodiments, the library of expression cassettes may be comprised within a plurality of plasmids. A plasmid is a small molecule of DNA within a cell that is physically separated from chromosomal DNA and can replicate independently. Plasmids are most commonly found as small, circular, double-stranded DNA molecules in bacteria, but are also found in archaea and eukaryotes. Artificial plasmids may be used as vectors in molecular cloning.

In some embodiments, a plurality of expression cassettes or a plurality of plasmids is provided. The plurality of expression cassettes or the plurality of plasmids may comprise 2-100 or more different expression cassettes or plasmids, respectively, wherein the number of different expression cassettes or plasmids within the plurality of expression cassettes or plasmids, respectively, is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more. In some embodiments, a plurality of expression cassettes or a plurality of plasmids may comprise at least five different expression cassettes or plasmids, respectively.

Artificially constructed plasmids may be used as vectors in genetic engineering and to clone and amplify or express genes of interest. Several plasmids are commercially available for such uses. The gene to be replicated is normally inserted into a plasmid that typically contains a number of features for their use. The features include: a gene that confers resistance to particular antibiotics (e.g. ampicillin); an origin of replication to allow the bacterial cells to replicate the plasmid DNA; and a suitable site for cloning. Yeast plasmids are similar to other, e.g. bacterial, plasmids in that they may contain a selection marker. Examples of available yeast plasmids include 2 μm plasmids, which are small circular plasmids often used for genetic engineering of yeast, and linear pGKL plasmids from Kluyveromyces lactis. Other plasmids that may be related to yeast cloning vectors include yeast integrative plasmid (YIp), and yeast replicative plasmid (YRp). YIp yeast vectors rely on integration into the host chromosome for survival and replication, and are usually used when studying the functionality of a solo gene or when the gene is toxic. YRp yeast vectors transport a sequence of chromosomal DNA that includes an origin of replication.

A plasmid cloning vector is typically used to clone DNA fragments of up to 15 kilobases. To clone longer lengths of DNA, lambda phage with lysogeny genes deleted, cosmids, bacterial artificial chromosomes, or yeast artificial chromosomes may be used.

Transformation is the genetic alteration of a cell resulting from the direct uptake and incorporation of exogenous genetic material, such as DNA, from its surroundings and taken up through the cell membrane(s). Transformation occurs naturally in some species of bacteria, but it can also be affected by artificial means in other cells. Transformation may be used to describe the insertion of new genetic material into nonbacterial cells, including animal, plant, and yeast cells. Most species of yeast, including Saccharomyces cerevisiae, as In some embodiments, may be transformed by exogenous DNA in the environment. Several methods have been developed to facilitate this transformation. Different yeast genera and species take up foreign DNA with different efficiencies, though most transformation protocols for yeast have been developed for S. cerevisiae.

Yeast cells may be treated with enzymes to degrade their cell walls, yielding spheroplasts, which are fragile but take up foreign DNA at a high rate.

Exposing intact yeast cells to alkali cations, such as those of cesium or lithium, lithium acetate, polyethylene glycol, or single-stranded DNA allows the cells to take up plasmid DNA. The single-stranded DNA preferentially binds to the yeast cell wall, preventing plasmid DNA from doing so and leaving it available for transformation.

Formation of transient holes in the cell membranes using electric shock or electroporation allows DNA to enter yeast cells, as in bacteria.

Enzymatic digestion or agitation with glass beads may also be used to transform yeast cells.

In some embodiments, the expression cassettes are flanked by sequences with sufficient identity to yeast chromosome sequences to permit transformation or integration of the expression cassette into the yeast genome.

In some embodiments, the expression cassettes or plasmids are assembled using Type IIS or “Golden Gate” cloning. Type IIS cloning systems take advantage of the unique properties of Type IIS restriction endonucleases, which cut dsDNA at a specified distance from the recognition sequence. Traditional Type II restriction enzymes bind and cut within palindromic sequences to create an overhang. Ligation of two such ends cut with the same enzyme will restore the restriction site. Type IIS enzymes bind asymmetric recognition elements and cut one or more bases outside of them, theoretically creating a seamless junction (without a scar). The use of Type IIS restriction endonucleases allows for the creation of custom overhangs, which is not possible with traditional restriction enzyme cloning. This type of cloning can be used to assemble multiple DNA fragments in any order, into any compatible vector, without scarring. The entire cloning step (digest and ligation) can be carried out in a single tube with a single restriction enzyme, since the resulting overhangs will be distinct and preserve the directionality of the cloning reaction. The restriction site is encoded on both the insert and plasmid in such a way that all recognition sequences are removed from the final product, with no resultant undesired sequence or scar. Type IIS cloning is useful in combinatorial assemblies, e.g. to test multiple promoters on a single transcription unit.

In some embodiments, libraries of expression cassettes are made by selecting promoter and terminator sequences for assembly into the expression cassettes by: limiting identity among sequences to less than 40 contiguous base pairs; varying promoter strengths determined by transcriptomics and expression data; including homologs to strong S. cerevisiae promoters from other yeasts; using expression-influencing terminators (including expression-enhancing terminators); using only promoter and terminator sequences from constitutive genes; and/or using promoter and terminator sequences that have no genome annotation describing known regulatory elements, open reading frames (ORFs), or centromeres; and assembling the selected promoter and terminator sequences into the expression cassettes.

In some embodiments, libraries of expression cassettes are made by selecting promoter and terminator sequences for assembly into the expression cassettes by: providing a plurality of promoter sequences, a plurality of terminator sequences, and a selection cassette sequence, wherein: the promoter sequences are flanked 5′ by a sequence that has identity with a sequence that is 5′ to an integration site on a yeast genome, and are flanked 3′ by a fragment of a detectable marker; the terminator sequences are flanked 5′ by an overlapping fragment of the detectable marker, wherein the two fragments of the detectable marker comprise sufficient sequence when combined to express a functional detectable marker, and are flanked 3′ by a sequence that has identity with a selection cassette sequence; and the selection cassette sequence is flanked 5′ by a sequence that has identity with a sequence that is 3′ to the terminator sequences, and is flanked 3′ by a sequence that has identity with a sequence that is 3′ to an integration site on a yeast genome, combining the promoter sequences, the terminator sequences and the selection cassette sequence to prepare different combinations of promoter sequences and terminator sequences with the selection cassette sequence, transforming the combinations of sequences into yeast cells, and recombining and integrating the combinations of sequences into the genome of the yeast cells via homologous recombination.

Transcriptomics is the study of the transcriptome. The transcriptome is the complete set of RNA transcripts that are produced by the genome, under specific circumstances or in a specific cell, using high-throughput methods, such as microarray analysis. Comparison of transcriptomes allows the identification of genes that are differentially expressed in distinct cell populations, or in response to different treatments.

A constitutive gene is a gene that is continually transcribed. In contrast, a facultative gene is transcribed when needed. A housekeeping gene is typically a constitutive gene that is transcribed at a relatively constant level.

A regulatory sequence is a segment of a nucleic acid molecule which is capable of increasing or decreasing the expression of specific genes within an organism. A regulatory element may include a promoter, an enhancer, or a terminator. A cis-regulatory element is a region of non-coding DNA that can regulate the transcription of nearby genes.

An open reading frame (ORF) is the part of a genetic reading frame that has the potential to code for a protein or peptide. An ORF is a continuous stretch of codons beginning with a start codon (typically ATG) and ending with a stop codon (typically TAA, TAG or TGA).

A centromere is the part of a chromosome that links sister chromatids. Spindle fibers attach to the centromere via the kinetochore during mitosis. The physical role of centromeres is to act as the site of assembly of the kinetochore. The kinetochore is a highly complex multiprotein structure that is responsible for events of chromosome segregation, so that it is safe for cell division to proceed to completion and for cells to enter anaphase.

A detectable marker may include a fluorescent protein or a colorimetric enzyme. Without limitation, examples include, green fluorescent protein (GFP), yellow fluorescent protein (YFP), blue fluorescent protein (BFP), cyan fluorescent protein (CYP), red fluorescent protein (RFP), β-galactosidase/lacZ, luciferase, β-lactamase, chloramphenicol acetyltransferase, or β-glucuronidase.

In some embodiments, assembling the selected promoter and terminator sequences into the expression cassettes is performed by providing a plurality of promoter sequences, a plurality of terminator sequences, and a selection cassette sequence.

In some embodiments, the promoter sequences, terminator sequences, and selection cassette sequences are polymerase chain reaction (PCR)-amplified sequences. Standard methods known in the art may be used for PCR amplification of sequences.

In some embodiments, a selection cassette sequence is chosen in combination with the promoter and terminator combinations, to tune gene expression. A selection cassette or gene cassette is a type of mobile genetic element that contains a gene and a recombination site. It may exist incorporated into an integron or as a free circular DNA. Gene cassettes or plasmids often carry antibiotic resistance (selection) genes, which in some embodiments are selected from two categories of selection cassettes: auxotrophic selection cassettes or antibiotic selection cassettes. In some embodiments, auxotrophic selection cassettes include HIS, LEU, URA, TRP, LYS, and MET cassettes and antibiotic selection cassettes include KanMX, NatMX, hphMX, and bleMX.

In some embodiments, a robotic or programmed liquid handler is used to combine the promoter, the terminator, and the selection cassette sequences. A robotic or programmed liquid handler comprises a class of devices that can include automated pipetting systems as well as microplate washers, that dispense and sample liquids in tubes or wells. These devices offer precision sample preparation for high throughput screening/sequencing (HTC), liquid or powder weighing, sample preparation, and bio-assays of many kinds.

In some embodiments, the design of synthetic yeast promoters comprises generating a nucleotide sequence of an upstream activation sequence 2 (UAS2), an upstream activation sequence 1 (UAS1), and a core comprising a TATA binding protein (TBP) region, a transcription start site (TSS), and a 5′ untranslated region (UTR).

In transcription, promoters are under the control of several elements. A DNA transcription unit encoding for a protein may contain a coding sequence, which is translated into protein, and regulatory sequences, which direct and regulate the synthesis of the protein. The regulatory sequence found upstream of the coding sequence and downstream of the promoter sequence is called the five prime untranslated region (5′UTR). The sequence found downstream of the coding sequence is called the three prime untranslated region (3′UTR).

An upstream activation sequence (UAS) or an upstream activating sequence is a cis-acting regulatory sequence or element. A UAS can increase the expression of an operably linked gene and plays an important role in activating transcription. Upstream activation sequences enhance the expression of a protein of interest through an increase in transcriptional activity. The upstream activation sequence is found adjacent to and upstream of a minimal promoter (TATA box) and serves as a binding site for transactivators. The transcriptional transactivator must bind to the UAS in the proper orientation for transcription to begin.

The TATA box is a cis-regulatory element usually found 25-30 base pairs upstream of the transcriptional start site (TSS) and upstream of the promoter region of genes. It is a binding site of either general transcription factors or histones and is involved in the process of transcription by RNA polymerase. During transcription, the TATA binding protein (TBP) normally binds to the TATA-box sequence, which unwinds the DNA and bends it through 80°. The AT-rich sequence of the TATA-box facilitates easy unwinding, due to weaker base-stacking interactions between A and T bases, as compared to between G and C.

In some embodiments, a synthetic yeast promoter is prepared by generating random nucleotide sequence of an upstream activation sequence 2 (UAS2), an upstream activation sequence 1 (UAS1), or a core comprising a TATA binding protein (TBP) region, a transcription start site (TSS), and a 5′ untranslated region (UTR). The nucleotide sequence is generated based on a predetermined expression strength and promoter element types that are included in the UAS2, UAS1, or core. Promoter element sequences can be substituted at predetermined locations in the UAS2, UAS1, or core to produce a synthetic UAS2 sequence, UAS1 sequence, or core sequence. The nucleotide sequence(s) then are synthesized and used to replace a part of a yeast promoter, such that one or more of the synthetic UAS2 sequence, the UAS1 sequence, and the core sequence replaces a part of a yeast promoter. In addition, in some embodiments, Type IIS restriction endonuclease recognition sequences, ATG sequences, and sequences that bind non-coding RNA degradation proteins (e.g., NAB3 and NRD1) can be removed from the random sequences and the promoter element sequences prior to synthesizing the nucleotide sequence. Examples of the generation of synthetic promoters is described in detail in Examples 6-10.

The present invention is further illustrated by the following Examples, which in no way should be construed as further limiting. The entire contents of all of the references (including literature references, issued patents, published patent applications, and related patent applications) cited throughout this application are hereby expressly incorporated by reference, in particular for the teachings that are referenced herein.

### EXAMPLES

### Example 1

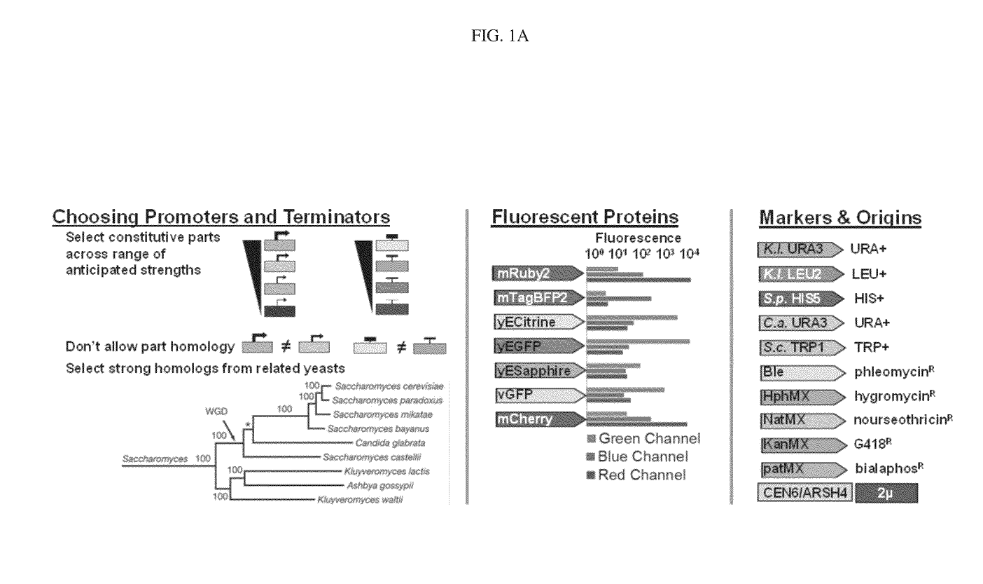

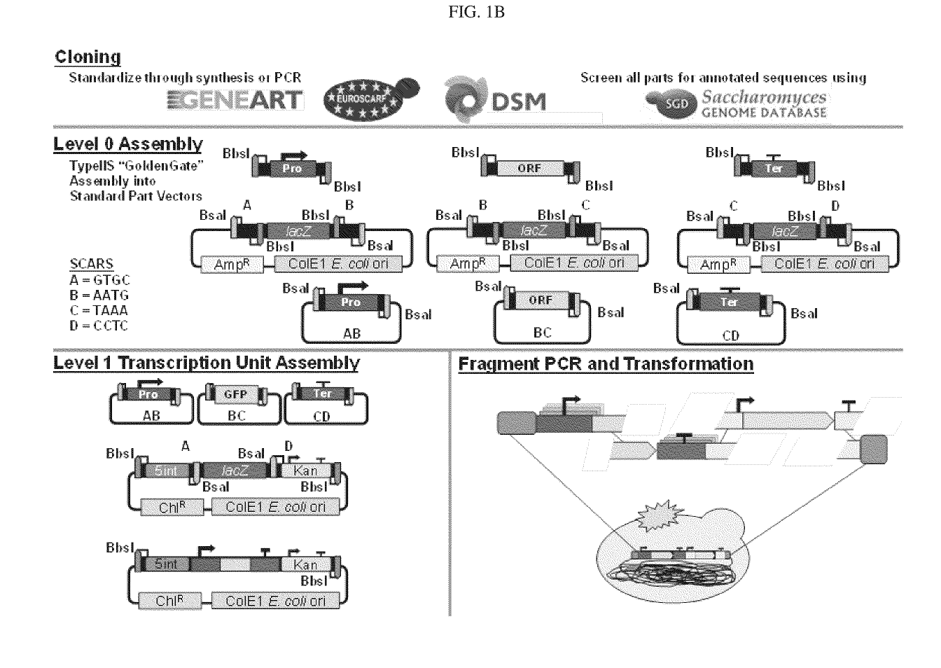

To select promoter and terminator sequences, the following guidelines were employed: (1) limit homology, (2) vary promoter strengths determined by published transcriptomics and GFP expression data, (3) import homologs to the strongest S. cerevisiae promoters from other yeasts, (4) use only expression-enhancing terminators, (5) all parts from constitutive genes, (6) clear annotation—no overlaps with known regulatory elements, ORFs, or centromeres (FIG. 1A).

The 38 promoters, 30 terminators, 7 fluorescent proteins, 10 selection markers, and 2 yeast origins of replication were standardized and selected using these guidelines. The promoters and terminators are listed in Table 1. The promoter sequences, terminator sequences, fluorescent protein sequences, and selection marker sequences can be found in the sequence listing.

Once selected and standardized, parts are cloned via a BbsI restriction-ligation into level 0 vector backbones in the first step of the Type IIS cloning process (FIG. 1B). To make the gene expression part characterization transcription units, a promoter, a terminator, and GFP are assembled into an expression cassette using a BsaI restriction-ligation. The Type IIS cloning site of the expression cassette destination vector is flanked by homology to chromosome XV of the S. cerevisiae genome. These vector sequences can be found in the sequence listing. It is essential to note that only one expression cassette needs to be made for each part, not every combination is constructed via Type IIS.

PCR amplification of the expression cassettes yields promoter fragments and terminator fragments. The promoter fragments possess homology 5′ to the integration site on the genome and a fraction of GFP. The terminator part fragments possess an overlapping fragment of GFP and homology to a NatMX selection cassette. The NatMX selection cassette also has homology to a PCR fragment with homology 3′ to the integration site on the genome. The primers for fragment amplification are listed in Tables 2A, 2B, 2C, and 2D. Using an acoustic liquid handler, thousands of unique combinations of promoters and terminators are made with these PCR-amplified part fragments. They are then transformed into yeast and combine via homologous recombination. In this way, an initial set of 38 promoters and 30 terminators were characterized, for a total of 1080 measurements. Successful integrations were cultured in CSM+Glucose+G418 for 16 hr and the fluorescence measured with flow cytometry.

### Example 2

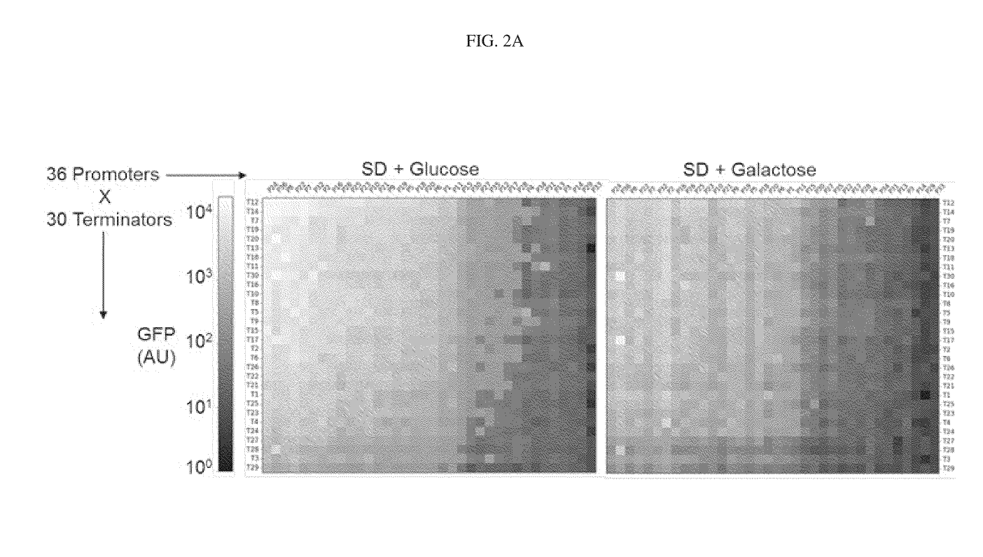

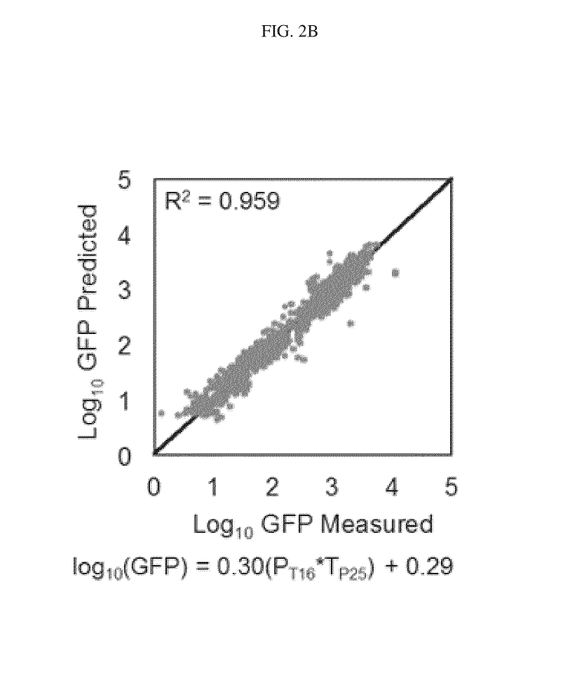

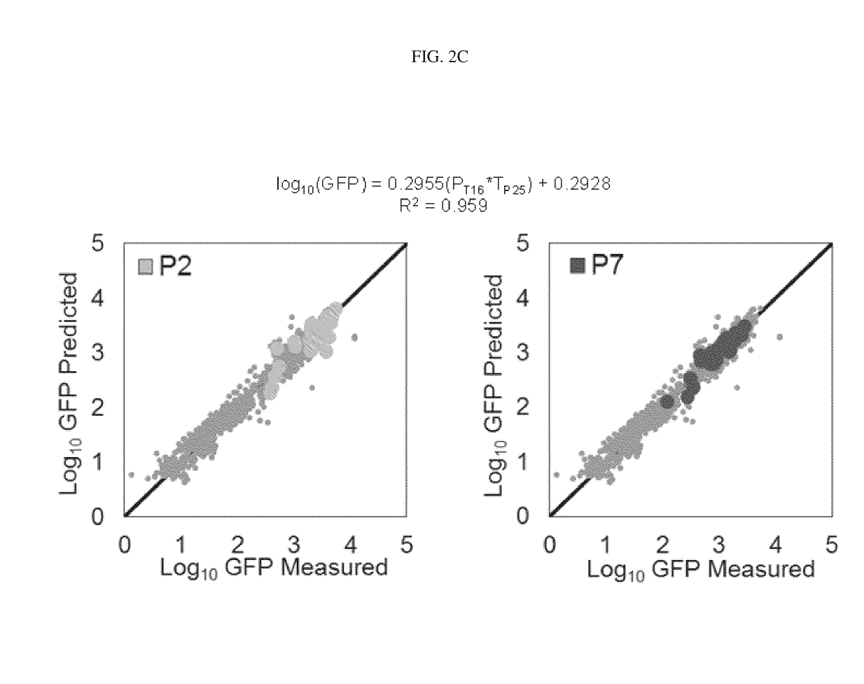

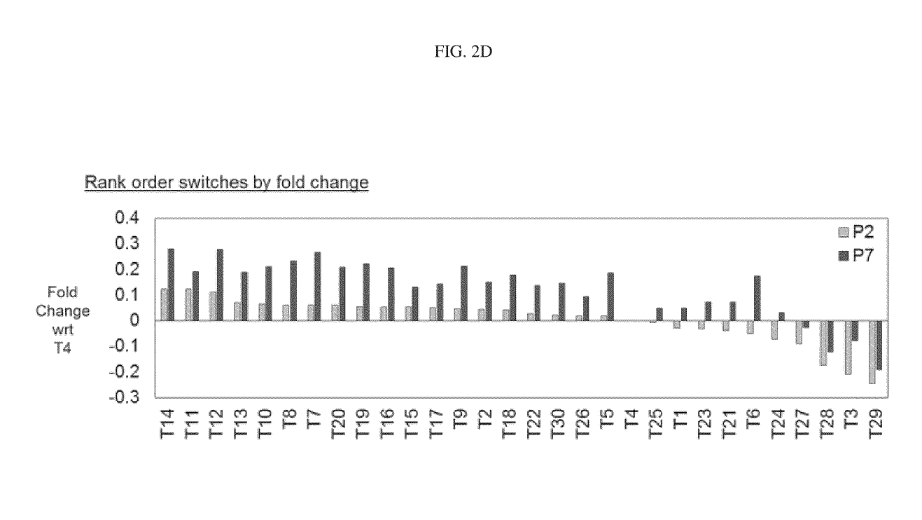

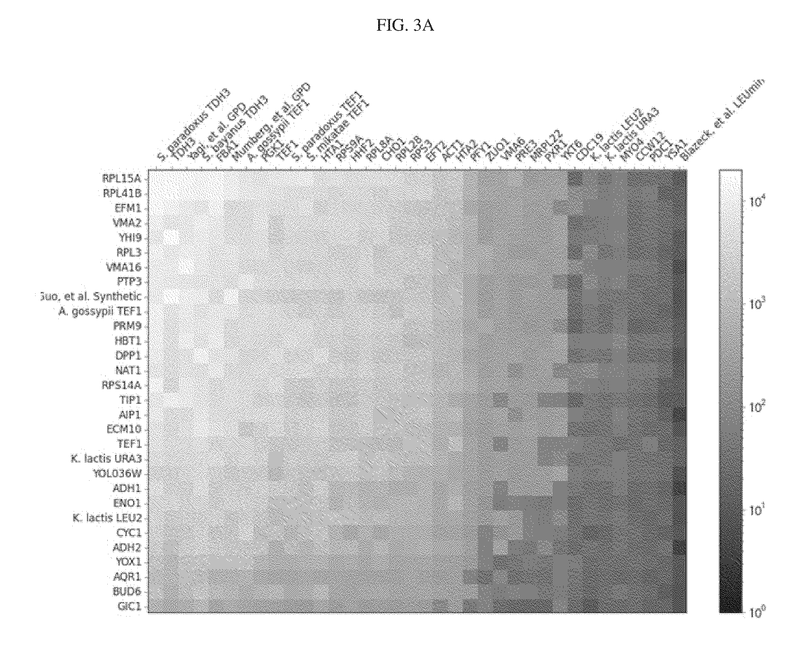

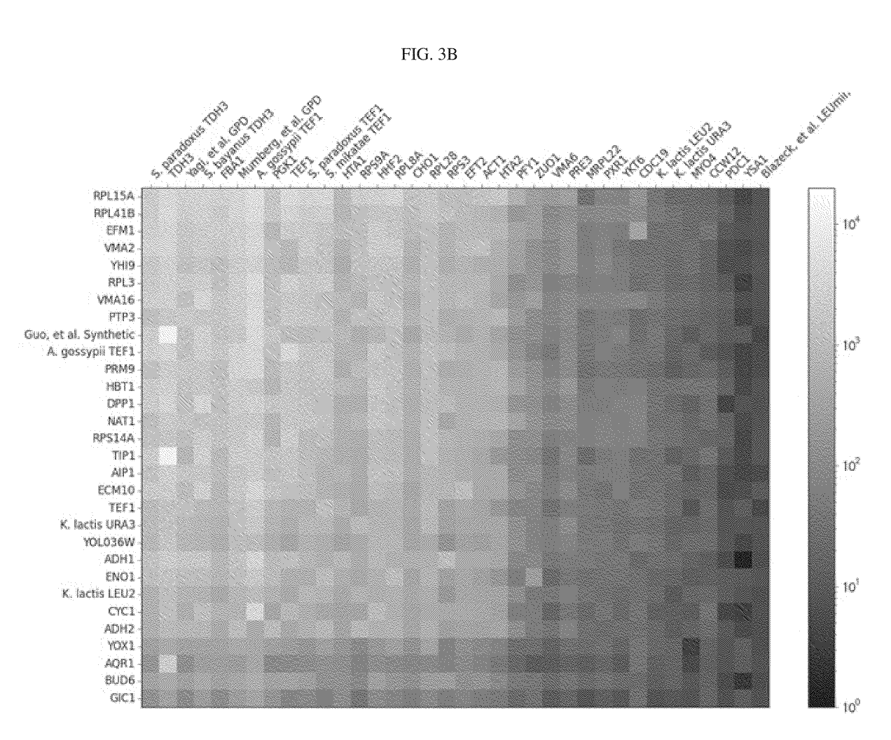

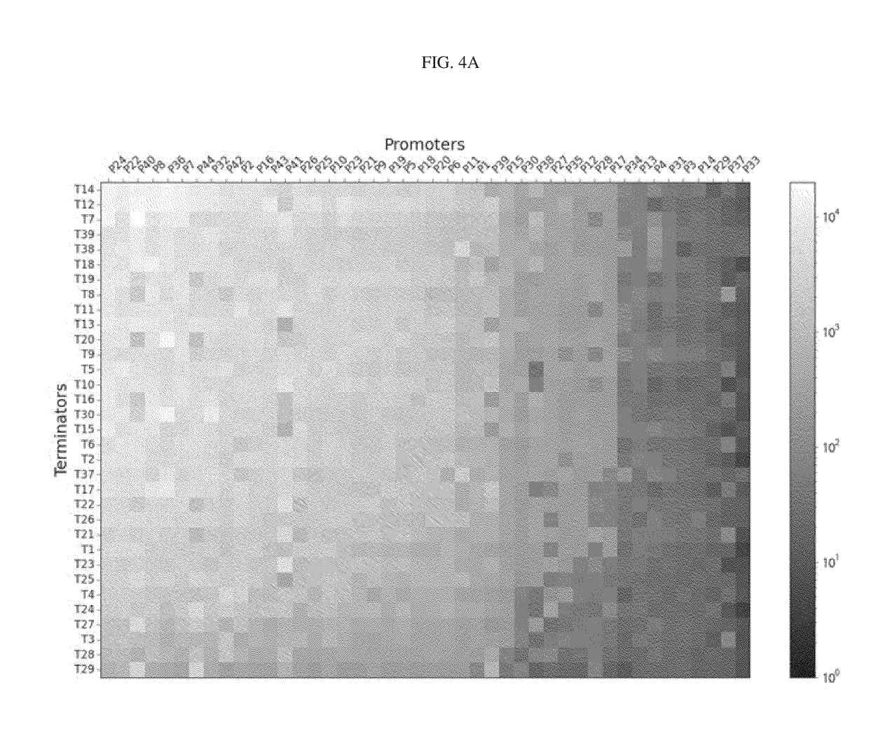

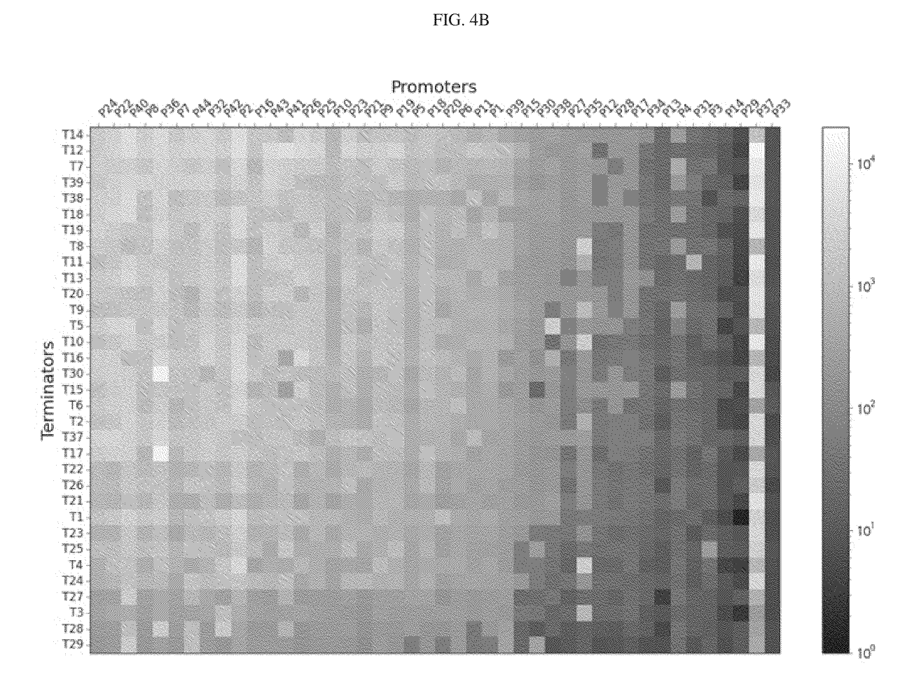

In the first characterization set, 1080 unique promoter-terminator combinations were constructed. FIGS. 2A, 3A, 3B, 4A, and 4B display a heatmap based on the autofluoresence-adjusted GFP expression level for the above combinations with glucose or galactose as the sole carbon source. Promoters are ranked by average expression level across all terminators in SD+glucose media, and terminators are ranked by average expression level across all promoters in SD+glucose media.

By appearance, this space seems well-behaved in that there is not a random distribution of strengths, i.e. expression-enhancing terminators are generally expression-enhancing across all promoters, etc. Therefore, we developed an empirical model to predict the expression of any promoter-terminator combination by using a small subset of the data. As inputs, we selected the fluorescence measurements associated with an individual representative promoter when paired with each of the terminators, as well as the measurements associated with a representative terminator when paired with each individual promoter. We regressed against all measured promoter-terminator combinations, and we found a simple linear relationship between the log-transformed fluorescence values. The model takes the form:

F(p,t)_{predicted}=c·F(p_{proxy},t)*F(p,t_{proxy})+k

Where F(p,t) is the log₁₀-transformed florescence for the combination of promoter p with terminator t. The F(p_{proxy},t) and F(p,t_{proxy}) are measured log 10-transformed florescence values measured for the query regulatory parts in the context of the proxy promoter and terminator respectively. The constants c and k are model parameters dependent on the selection of proxies and growth conditions. Next, to select the representative promoter and terminator, we repeated the regression calculation using all possible combinations of proxy promoters and terminators. We compared the model correlations and found that over 75% of the combinations produced models with R²>0.9. In order to select parameters for a general model, we selected P25 (S. paradoxus TEF1p) and T16 (A. gossypii TEF1t) because the pair produced high correlations in both glucose and galactose growth conditions (R²_{GLU}≈R²_{GAL}≈0.95). The model is shown in FIGS. 2B and 2C. FIG. 2D displays a comparison of P2 and P7, showing different expression levels between the two promoters across all terminators.

The predictive power of the model provides for a new way to design cassettes to express genes at target levels. The advantage of this approach is that it reduces the need to fully characterize all possible combinations of promoters and terminators. Rather, only a subset of parts are characterized. By characterizing the expression levels effected by all promoters (whether they be natural or synthetic) in the context of the representative terminator, and similarly by characterizing the expression levels effected by all terminators (whether they be natural or synthetic) in the context of the representative promoter, it is possible to use the model to predict all expression levels to within the error of the model. Thus by characterizing n promoters and m terminators, only n+m additional experiments need to be performed rather than all n×m experiments.

### Example 3

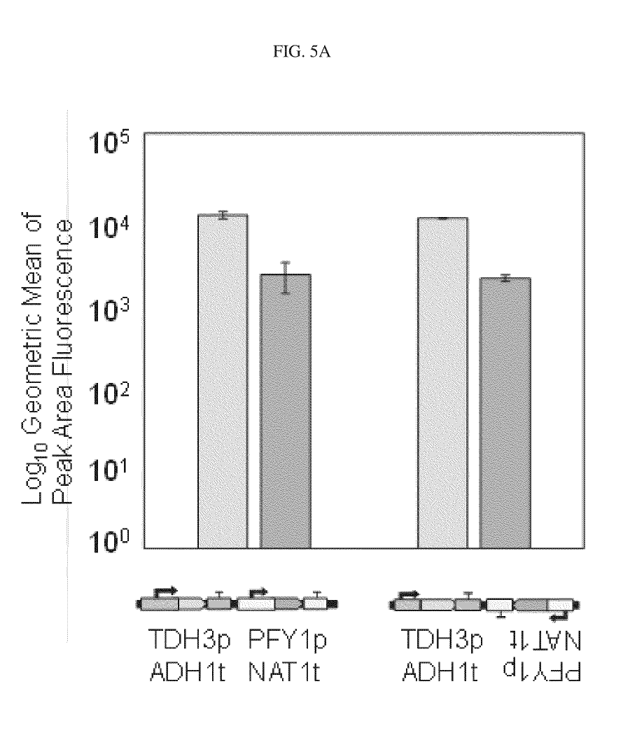

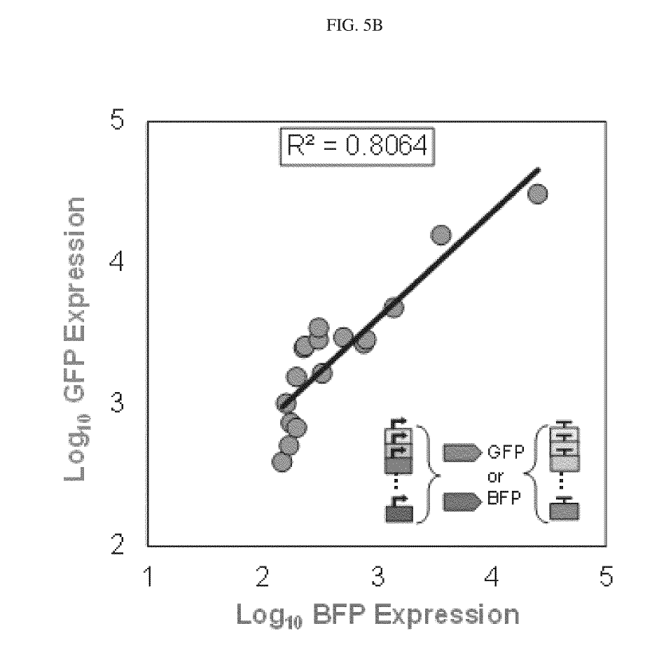

Part context effects. With the determination of expression strengths (FIGS. 2A-4B), and initial analysis of context effects or lack thereof (see model) (FIGS. 5A-5B), it is now possible to apply these precision gene control parts within genetic designs. These parts may be used in any context where expression control is necessary, such as controlling expression of one gene, either to overexpress or reduce expression due to toxicity, or in any synthetic circuit or metabolic engineering context where control is needed. In order to demonstrate the large scale enabled by these parts, we demonstrate the feasibility of constructing large libraries of genetic designs where particular levels of expression are required. These libraries particularly benefit from the standards, redundancy, and composability of the characterized parts.

### Example 4

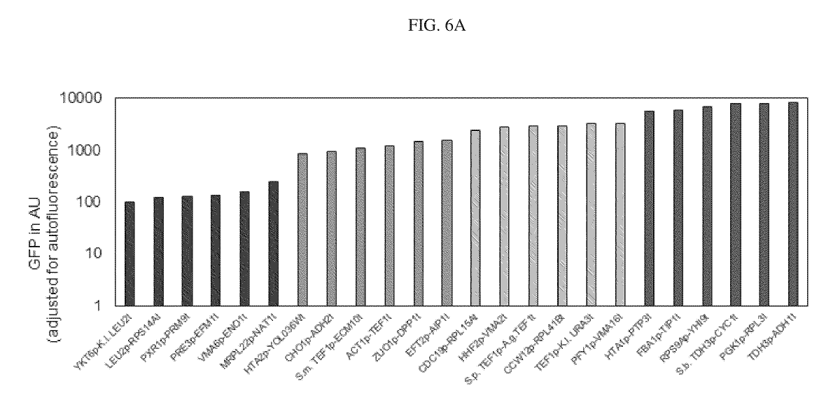

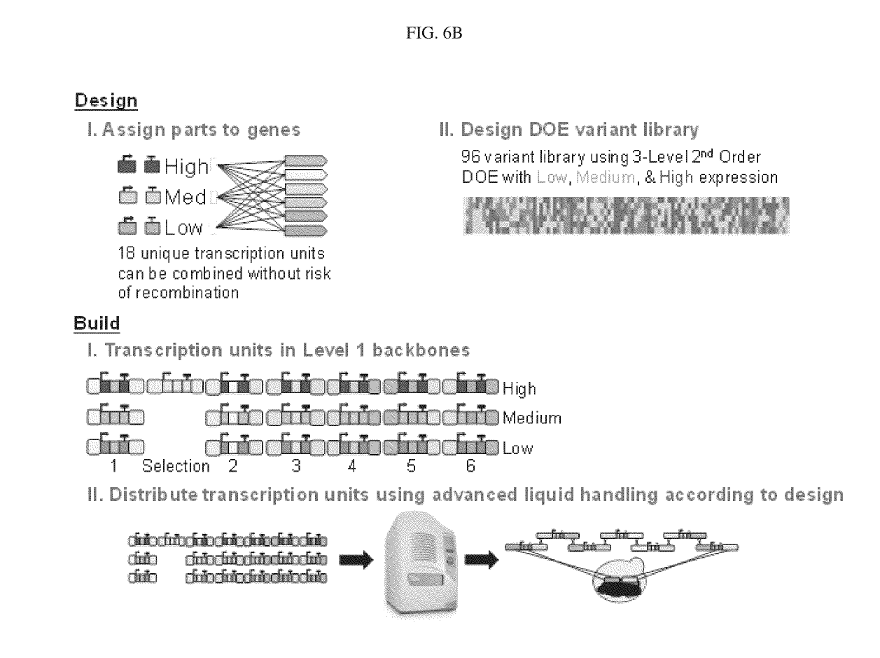

FIG. 6A depicts parts that can be chosen to have four redundant expression strengths for a six gene pathway. By assigning unique combinations to each pathway gene, any possible pathway permutation can be built without repeating any parts. Using this approach, a 192-variant combinatorial library of the six-gene itaconic acid pathway was constructed using Type IIS cloning and advanced liquid handling (FIG. 6B).

### Example 5

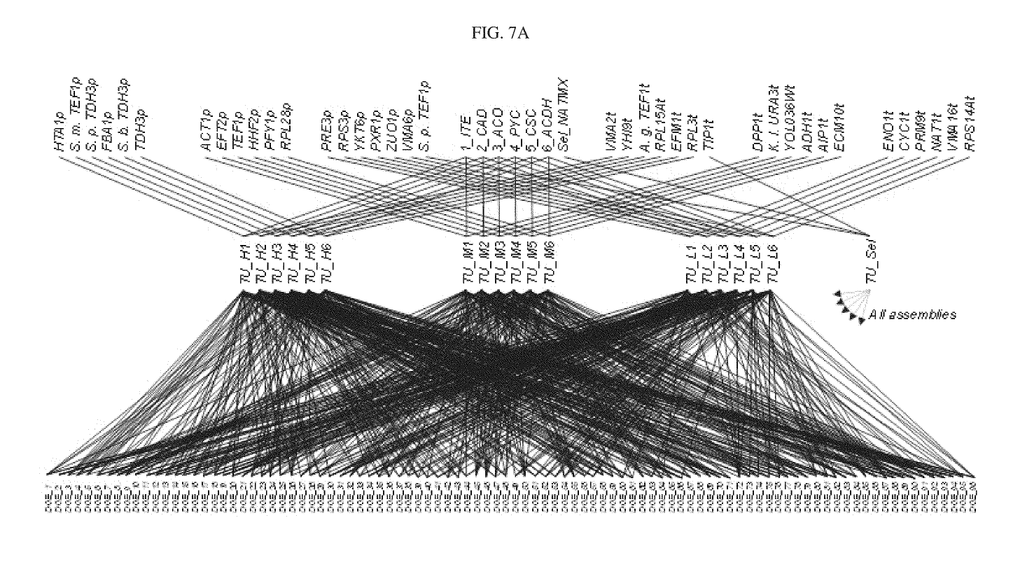

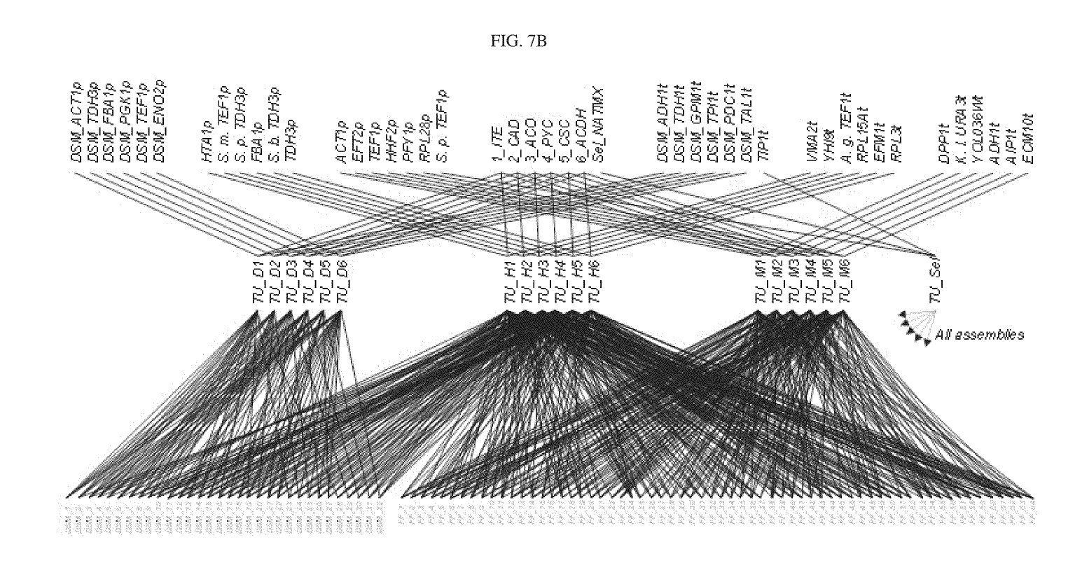

A pathway assembly strategy using promoter-terminator combinations was created to tune gene expression. First, parts were combined into transcription units according to their fit to predetermined expression levels, then the transcription units (expression cassettes) were combined into 192 pathway variants. FIG. 7A shows an assembly diagram of the hierarchical pathway assembly strategy enabled by the parts library. This set is a design-of-experiments library of 6 genes and 3 expression levels totaling 96 unique pathway designs. The top row shows all of the promoters, terminators, genes for the assembly. These are combined via Type IIS cloning into transcription units in the second row. The 18 transcription units are combined via liquid handling into the designs on the bottom. FIG. 7B shows an assembly diagram of the second 96 designs, assembled using the same method described in FIG. 7A. These have a different design strategy, however. The first 32 unique pathways combine in different patterns two sets of high strength promoter-terminator combinations. The other 64 designs are a full factorial set combining medium and high strength transcription units. The redundancy and predictability of the parts library are evident benefits in this context.

### Example 6

For large-scale synthetic promoter design, all known strength-enhancing binding sites and sequence features were combined into one high-throughput synthesis strategy, with sequence generation performed by a greedy constraint-based algorithm (ProGenie) for designing yeast promoters implemented in Python. This algorithm uses constraints on nucleotide content to design synthetic sequences, and then a further set of constraints to substitute various strength-enhancing sequence motifs, as shown in FIG. 8A. The algorithm is not computationally expensive, unlike design strategies based on nucleosome occupancy, and can thus design tens of thousands of promoter sequences in a matter of minutes. This is to produce a variety of different strength synthetic promoters.

The constraints on nucleotide content and motif substitution probability also change with the concept of “anticipated strength”. This is implemented as a set of four strength tiers in the algorithm, and the constraints on the sequence design are unique to each tier. Generally, motif substitution probability increases with increasing strength, graphically displayed in FIG. 8B.

The algorithm also incorporates a sequence editing functionality that removes undesired sequences that arise randomly and from substitution. There are three types of ‘undesired’ sequences in the algorithm. First are Type IIS sites that are used in subsequent cloning steps. Second are upstream ATG sites that may arise in the promoter near the start of the gene. It has been shown that upstream ATG sites dramatically decrease translational efficiency. Third are sequences that bind non-coding RNA degradation proteins NAB3 and NRD1. As many yeast promoters are naturally bidirectional, these signals exist as a way to rapidly degrade transcription initiated in the non-coding direction. However, if they arose in the synthetic sequences, it is likely that they would reduce the half-life of the resultant mRNAs, ultimately reducing the expression strength of the promoter.

A summary of the nucleotide percentage settings are listed in Table 3 and the motif substitution settings are listed in Table 4.

### Example 7

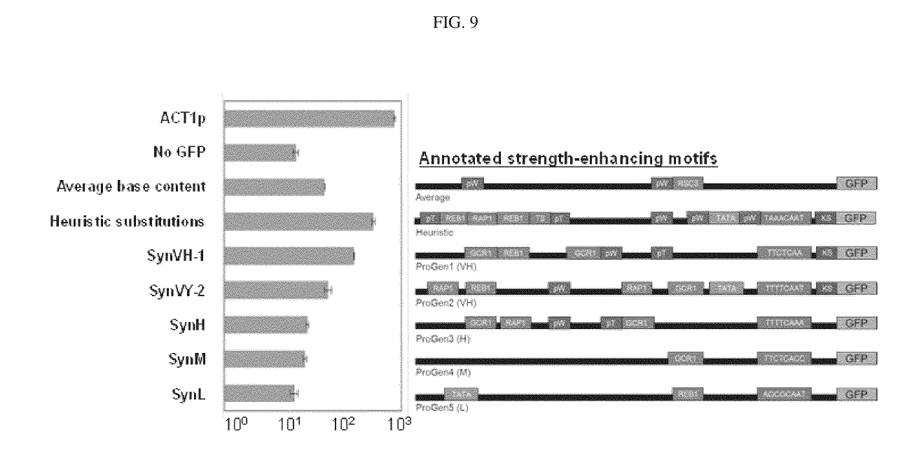

An initial set of promoters was designed using the ProGenie algorithm and compared against several controls: the native S. cerevisiae ACT1 promoter, random sequence with average yeast promoter nucleotide content, and a heuristic promoter designed with all of the highest-strength parameters incorporated. The data and motif annotation is shown in FIG. 9. Notably, the strength of each synthetic sequence matches its anticipated strength setting in the algorithm. Furthermore, it is also notable that simply creating random sequence is able to initiate transcription in yeast, and that the heuristic promoter is the strongest synthetic promoter. Sequences of the synthetic promoters in this proof-of-concept experiment are listed in the sequence listing.

### Example 8

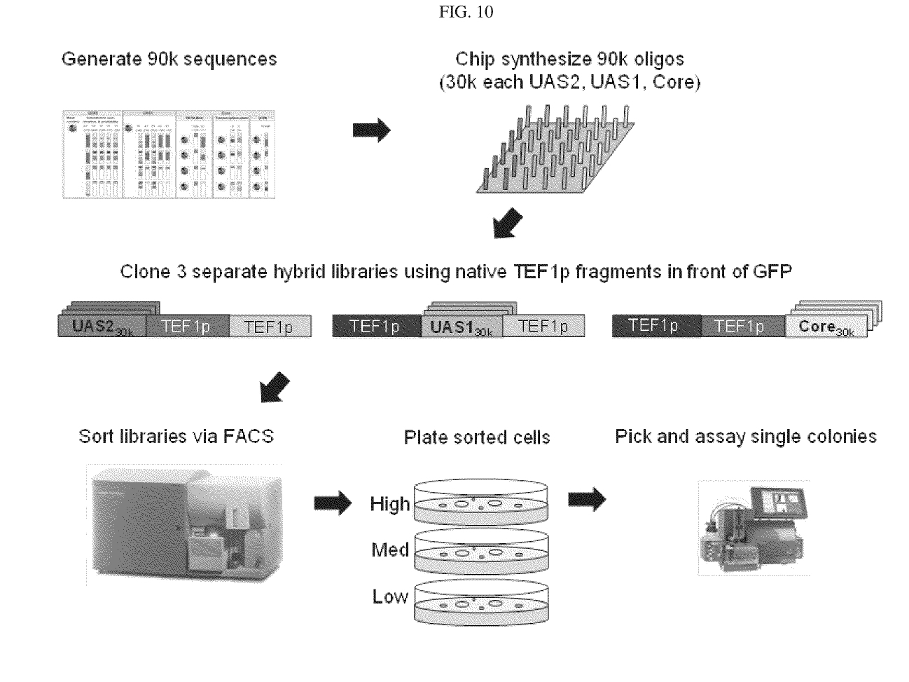

The initial data provides the basis for designing a high-throughput synthesis method to create thousands of synthetic promoters and search for functional sequences. Because of the limitations on oligo length for synthetic chip, segments of less than 150 base pairs are necessary. Since yeast promoters are much longer, a cloning strategy must be implemented to stitch the segments together after synthesis, as shown in FIG. 10. With this first synthetic oligo library, each segment was designed to replace a section of the native yeast TEF1 promoter. Thus, synthetic segments can be analyzed separately in the context of a native yeast promoter.

In this experiment, the different segments of synthetic sequences are combined with segments from the strong yeast TEF1 promoter. By cloning these three libraries in front of GFP, flow cytometry can be used to sort S. cerevisiae cells containing a synthetic promoter based on fluorescence intensity. Subsequent plating and sequencing of the cells in different strength bins can then provide insights into the elements that most influence transcriptional strength. FIG. 10 shows this workflow.

### Example 9

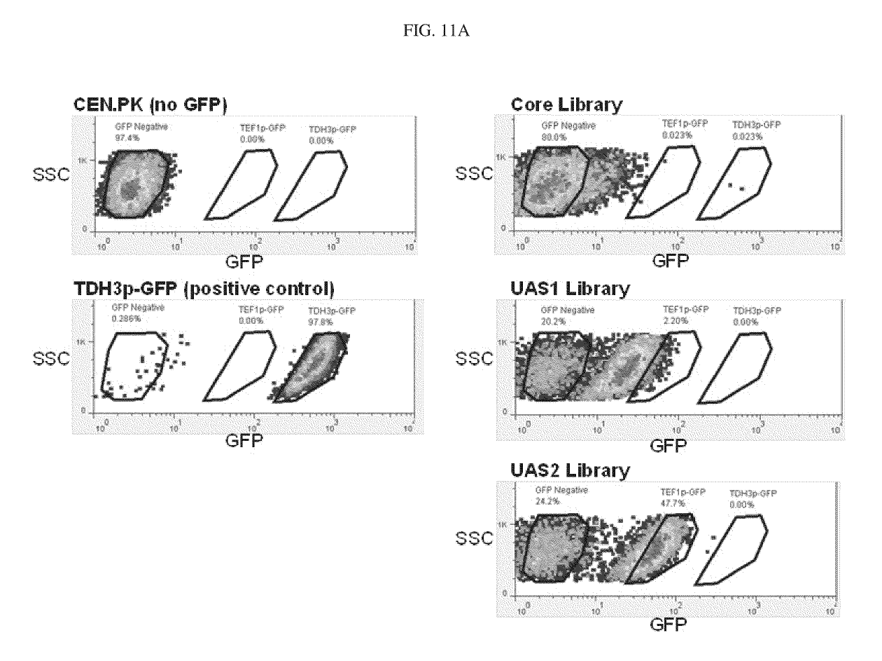

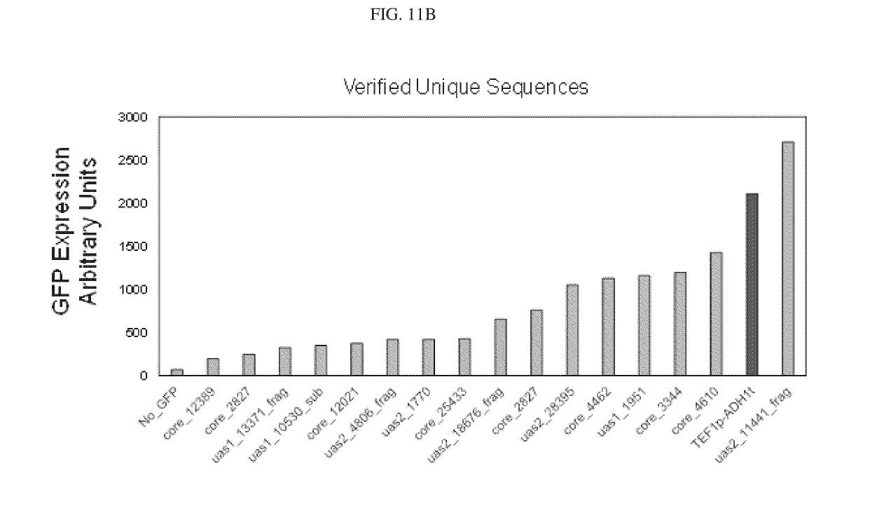

FIG. 11A shows plots of side scatter (SSC) versus GFP fluorescence for the synthetic promoter libraries and some controls. This visually displays the diversity and range of expression strengths achieved with 30 k synthetic sequences for each of the three promoter segments. The gates drawn on the plots are rough approximates of the actual gates used to sort the libraries. After plating, picking individual colonies, confirming activity via flow cytometry, and sequencing unique clones, 16 different unique sequences have been identified to date. The expression strength of each of these synthetic sequences is shown in FIG. 11B.

Next-generation sequencing will now be applied to the sorted bins to deep sequence thousands of variants, understanding and analysis of which promises to offer fundamental insights into transcriptional activation in S. cerevisiae.

Finally, with strong synthetic sequences isolated from this library, new synthetic promoters may be designed and implemented in large-scale genetic designs outlined within the description of this invention.

### Example 10

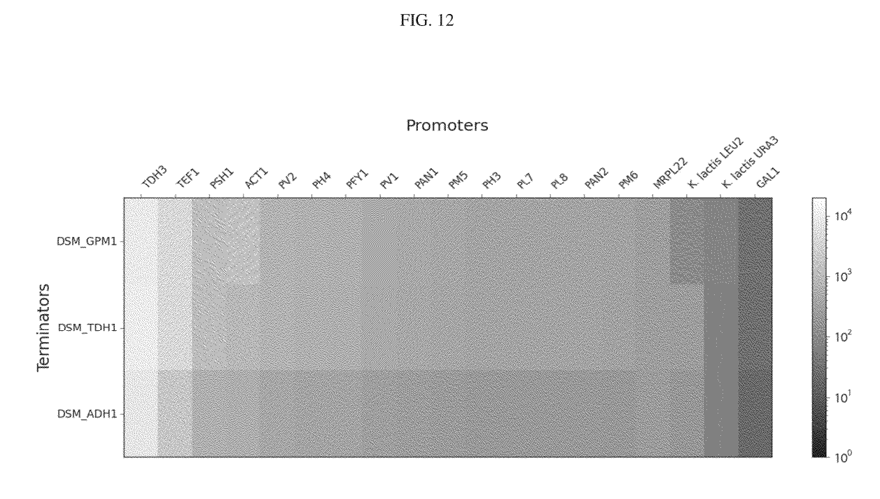

FIG. 12 shows a heatmap based on the autofluoresence-adjusted GFP expression level for combinations of synthetic promoters and reference promoters with three standard terminators, showing that designed synthetic yeast promoters may be used in combination with terminators to tune gene expression. The promoters span the medium range of activity and generally fall in the order of strength in which they were designed.

### TABLES

TABLE 1
Promoters and Terminators

| | | | | | | |
|---|---|---|---|---|---|---|
| | | | | S. cerevisiae | | |
| # | Genus | Species | Name | genome location | Citation | Length |

Promoters

| | | | | | | |
|---|---|---|---|---|---|---|
| P1 | Saccharomyces | cerevisiae | ACT1 | YFL039C | [15, 16] | 550 |
| P3 | Saccharomyces | cerevisiae | CCW12 | YLR110C | [15, 16] | 291 |
| P4 | Saccharomyces | cerevisiae | CDC19 | YAL038W | [15, 16] | 551 |
| P5 | Saccharomyces | cerevisiae | CHO1 | YER026C | [16, 17] | 550 |
| P6 | Saccharomyces | cerevisiae | EFT2 | YDR385W | [15, 16] | 551 |
| P7 | Saccharomyces | cerevisiae | FBA1 | YKL060C | [16] | 550 |
| P8 | Saccharomyces | cerevisiae | YagiGPD | — | [18] | 449 |
| P32 | Saccharomyces | cerevisiae | MumbergGPD | — | [19] | 654 |
| P9 | Saccharomyces | cerevisiae | HHF2 | YNL030W | [15, 16] | 548 |
| P10 | Saccharomyces | cerevisiae | HTA1 | YDR225W | [15, 16] | 551 |
| P11 | Saccharomyces | cerevisiae | HTA2 | YBL003C | [15, 16] | 550 |
| P33 | Saccharomyces | cerevisiae | LEU2 | YCL018W | [20, 21] | 122 |
| P34 | Kluyveromyces | lactis | LEU2 | — | [22] | 1024 |
| P12 | Saccharomyces | cerevisiae | MRPL22 | YNL177C | [16] | 453 |
| P13 | Saccharomyces | cerevisiae | MYO4 | YAL029C | [15, 16] | 552 |
| P14 | Saccharomyces | cerevisiae | PDC1 | YLR044C | [16] | 551 |
| P15 | Saccharomyces | cerevisiae | PFY1 | YOR122C | [16, 17] | 287 |
| P16 | Saccharomyces | cerevisiae | PGK1 | YCR012W | [6, 16] | 578 |
| P35 | Saccharomyces | cerevisiae | PRE3 | YJL001W | [16] | 599 |
| P17 | Saccharomyces | cerevisiae | PXR1 | YGR280C | [16] | 551 |
| P18 | Saccharomyces | cerevisiae | RPL28 | YGL103W | [15, 16] | 548 |
| P19 | Saccharomyces | cerevisiae | RPL8A | YHL033C | [15, 16] | 352 |
| P20 | Saccharomyces | cerevisiae | RPS3 | YNL178W | [15, 16] | 548 |
| P21 | Saccharomyces | cerevisiae | RPS9A | YPL081W | [15, 16] | 546 |
| P22 | Saccharomyces | bayanus | TDH3 | — | This study | 474 |
| P36 | Saccharomyces | cerevisiae | TDH3 | YGR192C | [16] | 599 |
| P24 | Saccharomyces | paradoxus | TDH3 | — | This study | 467 |
| P26 | Saccharomyces | cerevisiae | TEF1 | YPR080W | [16, 19] | 411 |
| P2 | Ashbya | gossypii | TEF1 | — | [22] | 378 |
| P23 | Saccharomyces | mikatae | TEF1 | — | This study | 410 |
| P25 | Saccharomyces | paradoxus | TEF1 | — | This study | 414 |
| P31 | Kluyveromyces | lactis | URA3 | — | [22] | 492 |
| P27 | Saccharomyces | cerevisiae | VMA6 | YLR447C | [16, 17] | 550 |
| P28 | Saccharomyces | cerevisiae | YKT6 | YKL196C | [16, 17] | 285 |
| P29 | Saccharomyces | cerevisiae | YSA1 | YBR111C | [16, 17] | 264 |
| P30 | Saccharomyces | cerevisiae | ZUO1 | YGR285C | [16] | 550 |
| P37 | Saccharomyces | cerevisiae | GAL1 | YBR020W | [23] | 600 |
| P38 | Saccharomyces | cerevisiae | CUP1 | YHR053C | [24] | 600 |

Terminators

| | | | | | | |
|---|---|---|---|---|---|---|
| T1 | Saccharomyces | cerevisiae | ADH1 | YOL086C | [16] | 101 |
| T24 | Saccharomyces | cerevisiae | ADH2 | YMR303C | [16] | 284 |
| T2 | Saccharomyces | cerevisiae | AIP1 | YMR092C | [5, 16] | 106 |
| T3 | Saccharomyces | cerevisiae | BUD6 | YLR319C | [7, 16] | 120 |
| T4 | Saccharomyces | cerevisiae | CYC1 | YJR048W | [16] | 216 |
| T5 | Saccharomyces | cerevisiae | DPP1 | YDR284C | [7, 16] | 172 |
| T6 | Saccharomyces | cerevisiae | ECM10 | YEL030W | [5, 16] | 213 |
| T7 | Saccharomyces | cerevisiae | EFM1 | YHL039W | [7, 16] | 75 |
| T25 | Saccharomyces | cerevisiae | ENO1 | YGR254W | [16] | 295 |
| T8 | Saccharomyces | cerevisiae | HBT1 | YDL223C | [7, 16] | 425 |
| T23 | Kluyveromyces | lactis | LEU2 | — | [22] | 137 |
| T9 | Saccharomyces | cerevisiae | NAT1 | YDL040C | [7, 16] | 136 |
| T10 | Saccharomyces | cerevisiae | PRM9 | YAR031W | [5, 16] | 249 |
| T11 | Saccharomyces | cerevisiae | PTP3 | YER075C | [7, 16] | 287 |
| T12 | Saccharomyces | cerevisiae | RPL15A | YLR029C | [7, 16] | 149 |
| T13 | Saccharomyces | cerevisiae | RPL3 | YOR063W | [7, 16] | 228 |
| T14 | Saccharomyces | cerevisiae | RPL41B | YDL133C-A | [7, 16] | 454 |
| T15 | Saccharomyces | cerevisiae | RPS14A | YCR031C | [7, 16] | 216 |
| T16 | Ashbya | gossypii | TEF1 | — | [22] | 239 |
| T26 | Saccharomyces | cerevisiae | TEF1 | YPR080W | [16] | 300 |
| T17 | Saccharomyces | cerevisiae | TIP1 | YBR067C | [5, 16] | 249 |
| T22 | Kluyveromyces | lactis | URA3 | — | [22] | 117 |
| T18 | Saccharomyces | cerevisiae | VMA16 | YHR026W | [7, 16] | 243 |
| T19 | Saccharomyces | cerevisiae | VMA2 | YBR127C | [7, 16] | 197 |
| T20 | Saccharomyces | cerevisiae | YHI9 | YHR029C | [7, 16] | 241 |
| T21 | Saccharomyces | cerevisiae | YOL036W | YOL036W | [5, 16] | 190 |
| T27 | Saccharomyces | cerevisiae | YOX1 | YML027W | [7, 16] | 400 |
| T28 | Saccharomyces | cerevisiae | AQR1 | YNL065W | [7, 16] | 350 |
| T29 | Saccharomyces | cerevisiae | GIC1 | YHR061C | [7, 16] | 225 |
| T30 | Saccharomyces | cerevisiae | GuoSynTer | — | [25] | 39 |

TABLE 2A
Primer Sequences for Promoter Fragment
Amplification
Template: pEMY11AD-PTdest-Pro-GFP-Ter Assembly

| | |
|---|---|
| EY520-F-63 | TTACCAATCCTTTCATAAGCTAATTATGCC |
| | (SEQ ID NO: 90) |
| EY632-R-65 | CATCTTCAATGTTGTGTCTAATTTTGAAGTTAGC |
| | (SEQ ID NO: 91) |

TABLE 2B
Primer Sequences for Terminator Fragment
Amplification
Template: pEMY11AD-PTdest-Pro-GFP-Ter Assembly

| | |
|---|---|
| EY633-R-65 | GTGCGGCCATCAAAATGTATGG (SEQ ID NO: 92) |
| EY634-F-65 | TTATGTTCAAGAAAGAACTATTTTTTTCAAAGATGACGG |
| | (SEQ ID NO: 93) |

TABLE 2C
Primer Sequences for NatMX Selection Fragment
Amplification
Template: pEMY11AD-P2-M7(NatMX)-T16

| | |
|---|---|
| EY635-F-66 | TACCCTCCTTGACAGTCTTGACG |
| | (SEQ ID NO: 94) |
| EY636-R-63 | CATAGTGTCGGGAACAGGTCATTCTAAAAAAAGTAAAA |
| | TAAAATTGGATGGCGGCGTTAG (SEQ ID NO: 95) |

TABLE 2D
Primer Sequences for 3′ Homology Fragment
Amplification
Template: S. cerevisiae CENPK-113 genomic DNA

| | |
|---|---|
| EY637-F-61 | cgattcgatactaacgccgccatccaATTTTATT |
| | TTACTTTTTTTAGAATGACCTGTTCC |
| | (SEQ ID NO: 96) |
| EY521-R-63 | TTGTGACCGCCCTGC (SEQ ID NO: 97) |

TABLE 3
ProGenie Nucleotide Percentage Settings

| Nucleotide Percentage | | | | | |
|---|---|---|---|---|---|
| Settings | | A | T | C | G |
| TBP | VH | 30 | 34 | 18 | 18 |
| | H | 32 | 36 | 16 | 16 |
| | M | 36 | 30 | 16 | 18 |
| | L | 34 | 30 | 18 | 18 |
| TSS | VH | 24 | 48 | 18 | 10 |
| | H | 32 | 38 | 16 | 14 |
| | M | 34 | 30 | 18 | 18 |
| | L | 36 | 28 | 18 | 18 |
| UTR | VH | 40 | 24 | 20 | 16 |
| | H | 44 | 22 | 18 | 16 |
| | M | 36 | 28 | 18 | 18 |
| | L | 30 | 34 | 18 | 18 |

| | | | | |
|---|---|---|---|---|
| UAS1 & UAS2 | 30 | 40 | 16 | 14 |

TABLE 4
ProGenie Motif Substitution Settings

| | | | Cumulative Probability of Substitution | | | |
|---|---|---|---|---|---|---|
| UAS2 | | | VH | H | M | L |
| 1 polyA:T | T13 | TTTTTTTTTTTTT | 0.9 | 0.75 | 0.5 | 0.1 |
| (AT) | | (SEQ ID NO: 109) | | | | |
| | MIX | TTAATTTAATTTT | 0.1 | 0.25 | 0.5 | 0.9 |
| | | (SEQ ID NO: 110) | | | | |
| | No Site | - | 0 | 0 | 0 | 0 |
| 4 | REB1_1 | TTACCCGT | 0.36 | 0.15 | 0.025 | 0.004 |
| Transcription | REB1_2 | CAGCCCTT | 0.04 | 0.15 | 0.075 | 0.036 |
| Factor | RAP1_1 | ACACCCAAGCAT | 0.27 | 0.16875 | 0.0375 | 0.003 |
| Binding Site | | (SEQ ID NO: 111) | | | | |
| (TF) | RAP1_2 | ACCCCTTTTTTAC | 0.03 | 0.05625 | 0.0375 | 0.027 |
| | | (SEQ ID NO: 112) | | | | |
| | GCR1_1 | CGACTTCCT | 0.27 | 0.16875 | 0.0375 | 0.003 |
| | GCR1_2 | CGGCATCCA | 0.03 | 0.05625 | 0.0375 | 0.027 |
| | No Site | — | 0 | 0.25 | 0.75 | 0.9 |
| | | | Cumulative Probability of Substitution | | | |
| UAS1 | | | VH | H | M | L |
| 3 polyA:T | T13 | TTTTTTTTTTTTT | 0.9 | 0.5625 | 0.125 | 0.01 |
| (AT) | | (SEQ ID NO: 109) | | | | |
| | MIX | TTAATTTAATTTT | 0.1 | 0.1875 | 0.125 | 0.09 |
| | | (SEQ ID NO: 110) | | | | |
| | No Site | — | 0 | 0.25 | 0.75 | 0.9 |
| 2 | REB1_1 | TTACCCGT | 0.225 | 0.125 | 0.046875 | 0.0125 |
| Transcription | REB1_2 | CAGCCCTT | 0.025 | 0.125 | 0.140625 | 0.1125 |
| Factor | RAP1_1 | ACACCCAAGCAT | 0.18 | 0.15 | 0.075 | 0.01 |
| Binding Site | | (SEQ ID NO: 111) | | | | |
| (TF) | RAP1_2 | ACCCCTTTTTTAC | 0.02 | 0.05 | 0.075 | 0.09 |
| | | (SEQ ID NO: 112) | | | | |
| | ABF1_1 | ATCATCTATCACG | 0.1 | 0.1 | 0.075 | 0.05 |
| | | (SEQ ID NO: 113) | | | | |
| | ABF1_2 | GTCATTTTACACG | 0.1 | 0.1 | 0.075 | 0.05 |
| | | (SEQ ID NO: 114) | | | | |
| | GCR1_1 | CGACTTCCT | 0.135 | 0.1125 | 0.05625 | 0.0075 |
| | GCR1_2 | CGGCATCCA | 0.015 | 0.0375 | 0.05625 | 0.0675 |
| | MCM1_1 | TTTCCGAAAACGGAA | 0.075 | 0.075 | 0.05625 | 0.0375 |
| | | AT (SEQ ID NO: 115) | | | | |
| | MCM1_2 | ATACCAAATACGGTA | 0.075 | 0.075 | 0.05625 | 0.0375 |
| | | AT (SEQ ID NO: 116) | | | | |
| | RSC3 | CGCGC | 0.05 | 0.05 | 0.0375 | 0.025 |
| | No Site | — | 0 | 0 | 0.25 | 0.5 |
| | | | Cumulative Probability of Substitution | | | |
| Core-TATA Binding Protein Region (TBP) | | | VH | H | M | L |
| 1 polyA:T | T13 | TTTTTTTTTTTTT | 0.75 | 0.375 | 0.0625 | 0.01 |
| (AT) | | (SEQ ID NO: 109) | | | | |
| | MIX | TTAATTTAATTTT | 0.25 | 0.375 | 0.1875 | 0.09 |
| | | (SEQ ID NO: 110) | | | | |
| | No Site | — | 0 | 0.25 | 0.75 | 0.9 |
| TATA Box | TATA_1 | TATAAAAA | 0.03125 | 0.03125 | 0.03125 | 0.03125 |
| Site Variant | TATA_2 | TATATAAA | 0.03125 | 0.03125 | 0.03125 | 0.03125 |
| (TATAWAWR) | TATA_3 | TATAAATA | 0.03125 | 0.03125 | 0.03125 | 0.03125 |
| | TATA_4 | TATATATA | 0.03125 | 0.03125 | 0.03125 | 0.03125 |
| | TATA_5 | TATAAAAG | 0.03125 | 0.03125 | 0.03125 | 0.03125 |
| | TATA_6 | TATATAAG | 0.03125 | 0.03125 | 0.03125 | 0.03125 |
| | TATA_7 | TATAAATG | 0.03125 | 0.03125 | 0.03125 | 0.03125 |
| | TATA_8 | TATATATG | 0.03125 | 0.03125 | 0.03125 | 0.03125 |
| | No Site | — | 0.75 | 0.75 | 0.75 | 0.75 |
| | | | Cumulative Probability of Substitution | | | |
| Core-Transcription Start Site (TSS) | | | VH | H | M | L |
| Upstream | U1 | TTTT | 0.2278 | 0.15 | 0.0625 | 0.067 |
| TSS Element | U2 | TTCT | 0.2211 | 0.15 | 0.0625 | 0.067 |
| | U3 | CTTA | 0.2211 | 0.15 | 0.0625 | 0.067 |
| | U4 | AGCG | 0 | 0.05 | 0.0625 | 0.469 |
| | No Site | — | 0.33 | 0.5 | 0.75 | 0.33 |
| TSS Element | E1 | CAAA | 0.335 | 0.2 | 0.0625 | 0.067 |
| | E2 | CAAT | 0.335 | 0.2 | 0.0625 | 0.067 |
| | E3 | CACC | 0 | 0.05 | 0.0625 | 0.268 |
| | E4 | ACAA | 0 | 0.05 | 0.0625 | 0.268 |
| | No Site | — | 0.33 | 0.5 | 0.75 | 0.33 |
| | | | Cumulative Probability of Substitution | | | |
| Core-5' Untranslated Region (UTR) | | | VH | H | M | L |
| Kozak Site | K1 | AAAAGTAAA | 0.475 | 0.2 | 0.0625 | 0.067 |
| Variant | K2 | AAAAACAAA | 0.475 | 0.2 | 0.0625 | 0.067 |
| | K3 | CCACCGGCG | 0 | 0.05 | 0.0625 | 0.268 |
| | K4 | CCACCAGTG | 0 | 0.05 | 0.0625 | 0.268 |
| | No Site | — | 0.05 | 0.5 | 0.75 | 0.33 |

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

### REFERENCES

1. Alper, H., et al., Tuning genetic control through promoter engineering. PNAS, 2006. 102(36): p. 12678-12683.
2. Wiedemann, B. and E. Boles, Codon-optimized bacterial genes improve L-arabinose fermentation in recombinant Saccharomyces cerevisiae. Applied and Environmental Microbiology, 2008. 74(7): p. 2043-2050.
3. Young, E. and H. Alper, Synthetic Biology: Tools to Design, Build, and Optimize Cellular Processes. Journal of Biomedicine and Biotechnology, 2010.
4. Blazeck, J. and H. S. Alper, Promoter engineering: Recent advances in controlling transcription at the most fundamental level. Biotechnology Journal, 2013. 8(1).
5. Curran, K. A., et al., Use of expression-enhancing terminators in Saccharomyces cerevisiae to increase mRNA half-life and improve gene expression control for metabolic engineering applications. Metab Eng, 2013. 19: p. 88-97.
6. Sun, J., et al., Cloning and characterization of a panel of constitutive promoters for applications in pathway engineering in Saccharomyces cerevisiae. Biotechnology and Bioengineering, 2012. 109(8): p. 2082-2092.
7. Yamanishi, M., et al., A Genome-Wide Activity Assessment of Terminator Regions in Saccharomyces cerevisiae Provides a “Terminatome” Toolbox. Acs Synthetic Biology, 2013. 2(6): p. 337-347.
8. Shalem, O., et al., Measurements of the Impact of 3′ End Sequences on Gene Expression Reveal Wide Range and Sequence Dependent Effects. Plos Computational Biology, 2013. 9(3).
9. Kosuri, S., et al., Composability of regulatory sequences controlling transcription and translation in Escherichia coli. Proceedings of the National Academy of Sciences of the United States of America, 2013. 110(34): p. 14024-14029.
10. Lee, M. E., et al., A Highly Characterized Yeast Toolkit for Modular, Multipart Assembly. ACS Synth Biol, 2015.
11. Weber, E., et al., A Modular Cloning System for Standardized Assembly of Multigene Constructs. Plos One, 2011. 6(2).
12. Redden, H. and H. S. Alper, The development and characterization of synthetic minimal yeast promoters. Nat Commun, 2015. 6: p. 7810.
13. Mogno, I., J. C. Kwasnieski, and B. A. Cohen, Massively parallel synthetic promoter assays reveal the in vivo effects of binding site variants. Genome Res, 2013.
14. Sharon, E., et al., Inferring gene regulatory logic from high-throughput measurements of thousands of systematically designed promoters. Nature Biotechnology, 2012. 30(6): p. 521-+.
15. Lubliner, S., L. Keren, and E. Segal, Sequence features of yeast and human core promoters that are predictive of maximal promoter activity. Nucleic Acids Research, 2013. 41(11): p. 5569-5581.
16. Holstege, F. C., et al., Dissecting the regulatory circuitry of a eukaryotic genome. Cell, 1998. 95(5): p. 717-28.
17. Blount, B. A., et al., Rational Diversification of a Promoter Providing Fine-Tuned Expression and Orthogonal Regulation for Synthetic Biology. Plos One, 2012. 7(3).
18. Yagi, S., et al., The UAS of the yeast GAPDH promoter consists of multiple general functional elements including RAP1 and GRF2 binding sites. J Vet Med Sci, 1994. 56(2): p. 235-44.
19. Mumberg, D., R. Muller, and M. Funk, Yeast vectors for the controlled expression of heterologous proteins in different genetic backgrounds. Gene, 1995. 156(1): p. 119-22.
20. Bitter, G. A., K. K. Chang, and K. M. Egan, A multi-component upstream activation sequence of the Saccharomyces cerevisiae glyceraldehyde-3-phosphate dehydrogenase gene promoter. Mol Gen Genet, 1991. 231(1): p. 22-32.
21. Guarente, L., et al., Distinctly regulated tandem upstream activation sites mediate catabolite repression of the CYC1 gene of S. cerevisiae. Cell, 1984. 36(2): p. 503-11.
22. Guldener, U., et al., A new efficient gene disruption cassette for repeated use in budding yeast. Nucleic Acids Res, 1996. 24(13): p. 2519-24.
23. Blazeck, J., et al., Controlling promoter strength and regulation in Saccharomyces cerevisiae using synthetic hybrid promoters. Biotechnology and Bioengineering, 2012. 109(11): p. 2884-2895.
24. Mascorro-Gallardo, J. O., A. A. Covarrubias, and R. Gaxiola, Construction of a CUP1 promoter-based vector to modulate gene expression in Saccharomyces cerevisiae. Gene, 1996. 172(1): p. 169-70.
25. Guo, Z. and F. Sherman, Signals sufficient for 3′-end formation of yeast mRNA. Mol Cell Biol, 1996. 16(6): p. 2772-6.
26. Lee, S., W. A. Lim, and K. S. Thorn, Improved blue, green, and red fluorescent protein tagging vectors for S. cerevisiae. PLoS One, 2013. 8(7): p. e67902.
27. Lam, A. J., et al., Improving FRET dynamic range with bright green and red fluorescent proteins. Nat Methods, 2012. 9(10): p. 1005-12.
28. Subach, O. M., et al., An enhanced monomeric blue fluorescent protein with the high chemical stability of the chromophore. PLoS One, 2011. 6(12): p. e28674.
29. Sheff, M. A. and K. S. Thorn, Optimized cassettes for fluorescent protein tagging in Saccharomyces cerevisiae. Yeast, 2004. 21(8): p. 661-70.
30. Gueldener, U., et al., A second set of loxP marker cassettes for Cre-mediated multiple gene knockouts in budding yeast. Nucleic Acids Res, 2002. 30(6): p. e23.
31. Goldstein, A. L., X. Pan, and J. H. McCusker, Heterologous URA3MX cassettes for gene replacement in Saccharomyces cerevisiae. Yeast, 1999. 15(6): p. 507-11.
32. Hegemann, J. H. and S. B. Heick, Delete and Repeat: A Comprehensive Toolkit for Sequential Gene Knockout in the Budding Yeast Saccharomyces cerevisiae, in Strain Engineering: Methods and Protocols, J. A. Williams, Editor. 2011, Springer Science and Business Media. p. 189-206.
33. Goldstein, A. L. and J. H. McCusker, Three new dominant drug resistance cassettes for gene disruption in Saccharomyces cerevisiae. Yeast, 1999. 15(14): p. 1541-53.
34. Campbell, M. K. e-Study Guide for Biochemistry 2012. p. 1-87.

1. A library of expression cassettes comprising
a plurality of expression cassettes, each comprising a promoter and a terminator;
wherein each of the promoters and terminators is different from all of the other promoters and terminators in the plurality of expression cassettes; and
wherein each of the promoters and terminators or each combination of a promoter and a terminator has a known or predicted expression strength.

2. The library of expression cassettes of claim 1, wherein the promoter and the terminator flank an insertion site for a nucleic acid molecule to be expressed.

3. The library of expression cassettes of claim 1, wherein each expression cassette of at least a first subset of the plurality of expression cassettes has about the same expression strength, optionally wherein each expression cassette of a second subset of the plurality of expression cassettes has about the same expression strength, which expression strength is different than the expression strength of the first subset of the plurality of expression cassettes.

4. (canceled)

5. The library of expression cassettes of claim 1, wherein one or more of the promoters are constitutive promoters, and/or wherein one or more of the promoters are synthetic promoters.

6. (canceled)

7. The library of expression cassettes of claim 1, wherein one or more of the terminators are expression-enhancing terminators, and/or wherein one or more of the terminators are synthetic terminators.

8. (canceled)

9. The library of expression cassettes of claim 1, wherein there is less than 40 bp contiguous identity between promoter sequences to prevent recombination, and/or wherein there is less than 40 bp contiguous identity between terminator sequences.

10. (canceled)

11. The library of expression cassettes of claim 1, wherein the expression cassettes are comprised within a plurality of plasmids.

12. The library of expression cassettes of claim 1, wherein the plurality of expression cassettes or the plurality of plasmids is at least 5 different expression cassettes or at least 5 different plasmids.

13. (canceled)

14. The library of expression cassettes of claim 1, wherein the expression cassette flanked by sequences with sufficient identity to yeast chromosome sequences to permit integration of the expression cassette into the yeast genome.

15. A method of making a library of expression cassettes comprising
selecting promoter and terminator sequences for assembly into the expression cassettes by (1) limiting identity among and between sequences to less than 40 bp contiguous identity; (2) varying promoter strengths determined by transcriptomics and expression data; (3) including homologs to strong S. cerevisiae promoters from other yeasts; (4) using expression-enhancing terminators; (5) using only promoter and terminator sequences from constitutive genes; and/or (6) using promoter and terminator sequences that have no genome annotation describing known regulatory elements, ORFs, or centromeres;
assembling the selected promoter and terminator sequences into the expression cassettes; and
measuring the expression strength of the expression cassettes or predicting the expression strength of the expression cassettes via a model, optionally wherein the model is an empirical model that predicts the expression of any promoter-terminator combination.

16. (canceled)

17. The method of claim 15, wherein the assembling the selected promoter and terminator sequences into the expression cassettes is performed by:
providing a plurality of promoter sequences, a plurality of terminator sequences, and a selection cassette sequence, wherein:
the promoter sequences are flanked 5′ by a sequence that has identity with a sequence that is 5′ to an integration site on a yeast genome, and are flanked 3′ by a fragment of a detectable marker;
the terminator sequences are flanked 5′ by an overlapping fragment of the detectable marker, wherein the two fragments of the detectable marker comprise sufficient sequence when combined to express a functional detectable marker, and are flanked 3′ by a sequence that has identity with a selection cassette sequence; and
the selection cassette sequence is flanked 5′ by a sequence that has identity with a sequence that is 3′ to the terminator sequences, and is flanked 3′ by a sequence that has identity with a sequence that is 3′ to an integration site on a yeast genome,
combining the promoter sequences, the terminator sequences, and the selection cassette sequence to prepare different combinations of promoter sequences and terminator sequences with the selection cassette sequence,
transforming the combinations of sequences into yeast cells, and
recombining and integrating the combinations of sequences into the genome of the yeast cells via homologous recombination.

18.-23. (canceled)

24. The method of claim 15, further comprising testing the expression of the detectable marker in the yeast cells to determine the expression strength of the combinations of the promoter and terminator sequences.

25. A method for constructing a genetic design comprising
selecting a plurality of expression cassettes from the library of claim 1, optionally wherein the plurality of expression cassettes is selected based on measuring the expression strength of the expression cassettes or predicting the expression strength of the expression cassettes via a model,
cloning an open reading frame sequence of the genetic design between the promoter and terminator sequences of each of the plurality of expression cassettes.

26.-27. (canceled)

28. The method of claim 25, wherein the genetic design is a genetic pathway or circuit, optionally wherein the genetic pathway or circuit is a metabolic pathway or a synthetic gene circuit.

29. (canceled)

30. The method of claim 25, wherein the cloning comprises assembling the promoter sequences, open reading frame sequences and terminator sequences in a yeast cell by homologous recombination, wherein:
the promoter sequences are flanked 5′ by a sequence that has identity with a sequence that is 5′ to an integration site on a yeast genome, and are flanked 3′ by a fragment of an open reading frame sequence;
the terminator sequences are flanked 5′ by an overlapping fragment of the open reading frame sequence, wherein the two fragments of the open reading frame sequence comprise sufficient sequence when combined to express a functional open reading frame sequence, and are flanked 3′ by a sequence that has identity with a selection cassette sequence; and
the selection cassette sequence is flanked 5′ by a sequence that has identity with a sequence that is 3′ to the terminator sequences, and is flanked 3′ by a sequence that has identity with a sequence that is 3′ to an integration site on a yeast genome,
optionally wherein the assembling comprises:
transforming the promoter sequences, open reading frame sequences and terminator sequences into yeast cells, and
recombining and integrating the promoter sequences, open reading frame sequences, and terminator sequences into the genome of the yeast cells via homologous recombination.

31.-32. (canceled)

33. A synthetic promoter comprising nucleotide sequences of anticipated strength and promoter element sequences,
wherein the nucleotide sequences of anticipated strength have nucleotide content that correlates with a predetermined expression strength;
wherein the promoter element sequences are selected for probable expression strength; and
wherein the nucleotide sequences of anticipated strength are interspersed with the promoter element sequences,
optionally wherein the nucleotide sequences of anticipated strength and promoter element sequences do not comprise Type IIS restriction endonuclease recognition sequences, ATG sequences, or sequences that bind non-coding RNA degradation proteins NAB3 and NRD1.

34.-35. (canceled)

36. A method of preparing a synthetic yeast promoter comprising
generating nucleotide sequences of an upstream activation sequence 2 (UAS2), an upstream activation sequence 1 (UAS1), and a core comprising a TATA binding protein (TBP) region, a transcription start site (TSS), and a 5′ untranslated region (UTR), wherein the nucleotide sequences satisfy constraints on the nucleotide sequences and are generated based on a predetermined expression strength and promoter element types that are included in the UAS2, UAS1, and core;
substituting promoter element sequences at predetermined locations in the UAS2, UAS1, and core, optionally wherein the promoter element sequences substituted at specific locations are selected from the group consisting of transcription factor binding site sequences, poly A/T sequences, TATA box sequences, transcription start element sequences, and Kozak element sequences; and
optionally synthesizing the nucleotide sequences.

37.-39. (canceled)

40. The method of claim 36, further comprising removing Type IIS restriction endonuclease recognition sequences, ATG sequences, and sequences that bind non-coding RNA degradation proteins NAB3 and NRD1 from the nucleotide sequences and the promoter element sequences prior to synthesizing the nucleotide sequences.

41. A method for preparing a synthetic yeast promoter comprising
generating nucleotide sequences of an upstream activation sequence 2 (UAS2), an upstream activation sequence 1 (UAS1), or a core comprising a TATA binding protein (TBP) region, a transcription start site (TSS), and a 5′ untranslated region (UTR), wherein the nucleotide sequences are generated based on a predetermined expression strength and promoter element types that are included in the UAS2, UAS1, or core;
substituting promoter element sequences at predetermined locations in the UAS2, UAS1, or core to produce a synthetic UAS2 sequence, UAS1 sequence, or core sequence, optionally wherein the synthetic UAS2 sequence, UAS1 sequence, or core sequence are a plurality of synthetic sequences and wherein replacing the part of the yeast promoter with one or more of the plurality of synthetic UAS2 sequences, the plurality of UAS1 sequences, and the plurality of core sequences produces a library of synthetic yeast promoters having one or more of the UAS2, UAS1, and core sequences replaced;
synthesizing the nucleotide sequences; and
replacing a part of a yeast promoter with one or more of the synthetic UAS2 sequence, the UAS1 sequence, and the core sequence.

42. (canceled)

43. The method of claim 41, further comprising removing Type IIS restriction endonuclease recognition sequences, ATG sequences, and sequences that bind non-coding RNA degradation proteins NABS and NRD1 from the random sequences and the promoter element sequences prior to synthesizing the nucleotide sequences.

44.-48. (canceled)


SEQUENCE LISTING (116 sequences)

SEQ ID NO: 1 - DNA, 550 bp, Saccharomyces cerevisiae
aaaatgtgtggggaagcgggtaagctgccacagcaattaatgcacaacatttaacctacattcttccttatcggatcctcaaaacccttaaaaacatatgcctcaccctaacatattttccaattaaccctcaatatttctctgtcacccggcctctattttccattttcttctttacccgccacgcgtttttttctttcaaatttttttcttctttcttctttttcttccacgtcctcttgcataaataaataaaccgttttgaaaccaaactcgcctctctctctcctttttgaaatatttttgggtttgtttgatcctttccttcccaatctctcttgtttaatatatattcatttatatcacgctctctttttatcttcctttttttcctctctcttgtattcttccttcccctttctactcaaaccaagaagaaaaagaaaaggtcaatctttgttaaagaataggatcttctactacatcagcttttagatttttcacgcttactgcttttttcttcccaagatcgaaaatttactgaattaac

SEQ ID NO: 2 - DNA, 378 bp, Ashbya gossypii
agcttgccttgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaac

SEQ ID NO: 3 - DNA, 291 bp, Saccharomyces cerevisiae
ttcgcggccacctacgccgctatctttgcaacaactatctgcgataactcagcaaattttgcatattcgtgttgcagtattgcgataatgggtgtcttacttccaacataacggcagaaagaaatgtgagaaaattttgcatcctttgcctccgttcaagtatataaagtcggcatgcttgataatctttctttccatcctacattgttctaattattcttattctcctttattctttcctaacataccaagaaattaatcttctgtcattcgcttaaacactatatcaat

SEQ ID NO: 4 - DNA, 551 bp, Saccharomyces cerevisiae
tccagaaactggcacttgacccaactctgccacgtgggtcgttttgccatcgacagattgggagattttcatagtagaattcagcatgatagctacgtaaatgtgttccgcaccgtcacaaagtgttttctactgttctttcttctttcgttcattcagttgagttgagtgagtgctttgttcaatggatcttagctaaaatgcatattttttctcttggtaaatgaatgcttgtgatgtgttccaagtgatttcctttccttcccatatgatgctaggtacctttagtgtgttcctaaaaaaaaaaaaaggctcgccatcaaaacgatattcgttggcttttttttctgaattataaatactctttggtaacttttcatttccaagaacctcttttttccagttatatcatggtcccctttcaaagttattctctactctttttcatattcattctttttcatcctttggttttttattcttaacttgtttattattctctcttgtttctatttacaagacaccaatcaaaacaaataaaacatcatcac

SEQ ID NO: 5 - DNA, 550 bp, Saccharomyces cerevisiae
ataaacaaaaatacgtgacccaaatactgtatacaccattgcaatagatatgattatagagcttatagctacatctttttagataaaagcgaagatgtttctgcgatttttccattatagctctccatgatactaaatatcaaggtctacatgtaagtatttgtatatatgggttggaatgtatatacgtatatacgtacgtacgtacgtatatgcacataattgttacgggatgtatatataaattagtagcattatagaagatatccctaacatcaatccccactccttctcaatgtgtgcagacttctgtgccagacactgaatatatatcagtaattggtcaaaatcactttgaacgttcacacggcaccctcacgcctttgagctttcacatggacccatctaaagatgaagatccgtattttataggaaacattataaataaggaaagagagatacacctatttttttcattttgtgggtgattgtcatttttagttgtctatttgattcaatcaaaaaacaaaaataaaactatatattaaaa

SEQ ID NO: 6 - DNA, 551 bp, Saccharomyces cerevisiae
ttccttttttttcttttttttttaccagaagaattacgtacaaaagtacctactatttcaaagcaagaaatgagatgcctattgtggttatatacagaatagatgataaatgggttttccgtgcaaaacgatatggagaattcaaaatgggtgcgaaatacctggaacgtaagcgttctgagaaatacacagacgcattaacctgacaaaaacacaactagtttgggaaagggatttggtctttcctctcgggcttctcgtgtggttcctttctttctcagatctccctgcacactgggctgttgtcctccaggttatggtttgttctcttcaggtattacaatgcagtaggcttttggagtgagcaaaacgaagagagaaaaaaattttttcttaaaagttttttttcattttgtgagcttattcttcttttctatatattcttgatatcttagattatacatattattctcttacatttcacgattgcccttttggtgtttagcattcagtctcaaagaccacaaacacaaactataacataattgcaa

SEQ ID NO: 7 - DNA, 550 bp, Saccharomyces cerevisiae
atgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggtgtcacgctgccgtgagcatcctctctttccatatctaacaactgagcacgtaaccaatggaaaagcatgagcttagcgttgctccaaaaaagtattggatggttaataccatttgtctgttctcttctgactttgtctcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaa

SEQ ID NO: 8 - DNA, 449 bp, Saccharomyces cerevisiae
cttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaatcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccagaacttagtttcgacgga

SEQ ID NO: 9 - DNA, 548 bp, Saccharomyces cerevisiae
acaagaagcaacgcgagagagcacaacacgctgttatcacgcaaactatgttttgacaccgagccatagccgtgattgtgcgtcacattgggcgataatgaacgctaaatgaccaactcccatccgtaggagccccttagggcgtgccaatagtttcacgcgcttaatgcgaagtgctcggaacggacaactgtggtcgtttggcaccgggaaagtggtactagaccgagagtttcgcatttgtatggcaggacgttctgggagcttcgcgtctcaagctttttcgggcgcgaaatgcagaccagaccagaacaaaacaactgacaagaaggcgtttaatttaatatgttgttcactcgcgcctgggctgttgttattcggctagatacatacgtgtttgtgcgtatgtagttatatcatatataagtatattaggatgaggcggtgaaagagattttttttttttcgcttaatttattcttttctctatcttttttcctacatcttgttcaaaagagtagcaaaaacaacaatcaatacaataaaat

SEQ ID NO: 10 - DNA, 551 bp, Saccharomyces cerevisiae
aaagggtgcaacgcgcgaaaaagtgagaacagccttccctttcgggcgacattgagcgtctaaccatagttaacgacccaaccgcgttttcttcaaatttgaactcgccgagctcacaaataattcattagcgctgttccaaaattttcgcctcactgtgcgaagctattggaatggagtgtatttggtggctcaaaaaaagagcacaatagttaactcgtcgttgttgaagaaacgcccgtagagatatgtggtttctcatgctgttatttgttattgcccactttgttgatttcaaaatcttttctcacccccttccccgttcacgaagccagccagtggatcgtaaatactagcaataagtcttgacctaaaaaatatataaataagtctcctaatcagcttgtagattttctggtcttgttgaaccatcatctatttacttccaatctgtacttctcttcttgatactacatcatcatacggatttggttatttctcagtgaataaacaacttcaaaacaaacaaatttcatacatataaaatataa

SEQ ID NO: 11 - DNA, 550 bp, Saccharomyces cerevisiae
tatttaaaaacctgtgttatgctcaaataacggttactgatccaaaaccttatatatgacggcaagtgtctcactgttgcattacgcgttgtttcttttctttgttcttgtaagcgcgattttaccagaactagatggcgctcgtgatcctgaaacggggagaaattttgagaacaccgctttattaggcgaagcggtgggcacagctcacgcgtaaggtgttcccattatttctcaaagtgatgcgaatttcagagaacacattaacctgggggccataaacgcgacgtgctaccattttcgttacgtatacttaggccagagattacaacatgactactaatatcaaacataactctatatataagggatgaagatgtatgctttcttagaatttcaaacatgttccgttaaagttttacttttcgatttcaatttcgactgcatgatgcttttcttagagagtgttttgttattaaatagtatcataaattcttgtctttttacataagaattaggaaagtacagaacaagagcaaatttaatatat

SEQ ID NO: 12 - DNA, 453 bp, Saccharomyces cerevisiae
cgatgacttcagtctattagtatgatgtttactaattaagcaaaatcagctcttgctgaataacactactattaaaaatcataaaaacatctttattgccaatagtgaagaacgacaaggccttttaaaaggcataaaaaacaatgataccattctgcaatgaaaaaaaaagaaaactttgttctttgtgtataaagaaaatattttagttctcctaaataaataaatactaaattatacgaaactctttcagtcttttctcctgcttgctgcttgaacagtactcatgatattctattaatcttctgtgggcggggtaactaagccttcactgtactcgggattttgaataaaagtcaactatccaatctggaaggcattttaacatacgccattaggagccgcatagcaagtcagagaacctcacctcaccctatcttttttattaaagaa

SEQ ID NO: 13 - DNA, 552 bp, Saccharomyces cerevisiae
ataacaatagaacaatagctttttgaccttccccttattttatttcaaaaggtaacagttagggctattcttaaactttctttagggtacacaatgaaaacatcaagattgagttaaaacccttaaaataaaactaggtctttaagaaactcactctccgggttatccatagaatgtttaccattctttagggttctgaactaatgcgaaaaaaaaaaaaggaaaagactgaaaaattcgaaaatatctcgaagtttagcagtagtagatgagaatagggtgtcttttatcgaaaacctgcaattgtaatacaacctttttttgaaagtcagcctttattctgattctgcatactcaattcctcaattcctacggagttatcacctttttttttaccttctcttcttttcttattgttctagctgaaaacattgttaccagtttggcgagacaatttattttcaatacgatacccttttgttcttctttttataattcaatctaattctaaaacacaaaaaaacaaaaaaaatcctataaccagttctcccg

SEQ ID NO: 14 - DNA, 551 bp, Saccharomyces cerevisiae
tttttgttgcctggtggcatttgcaaaatgcataacctatgcatttaaaagattatgtatgcacttctgacttttcgtgtgatgaggctcgtggaaaaaatgaataatttatgaatttgagaacaattttgtgttgttacggtattttactatggaataatcaatcaattgaggattttatgcaaatatcgtttgaatatttttccgaccctttgagtacttttcttcataattgcataatattgtccgctgcccctttttctgttagacggtgtcttgatctacttgctatcgttcaacaccaccttattttctaactattttttttttagctcatttgaatcagcttatggtgatggcacatttttgcataaacctagctgtcctcgttgaacataggaaaaaaaaatatataaacaaggctctttcactctccttgcaatcagatttgggtttgttccctttattttcatatttcttgtcatattcctttctcaattattattttctactcataacctcacgcaaaataacacagtcaaatcaatcaa

SEQ ID NO: 15 - DNA, 287 bp, Saccharomyces cerevisiae
aggagacgttactttgtttatatatattagtatgtacaatcgcaaagaaatggagtgatgacatgttgtagtatttagtatgaggttactgtgtgggaggttttaccatgatttttggcgagaacacgccatgaaatgtctttgtacgaaactcattacccgcattaatattttttttctttttaaagctcagttgaccctttctcattcccttcttaaaacaactgtgtgatccttgagaaaagataaattacatacacaacataaacccaactacgatcgcaaat

SEQ ID NO: 16 - DNA, 578 bp, Saccharomyces cerevisiae
tccctccttcttgaattgatgttaccctcataaagcacgtggcctcttatcgagaaagaaattaccgtcgctcgtgatttgtttgcaaaaagaacaaaactgaaaaaacccagacacgctcgacttcctgtgttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaac

SEQ ID NO: 17 - DNA, 551 bp, Saccharomyces cerevisiae
taaaaacgcagcaactaaagaaggcgactaataaaaattgagtatgcctctccttagcgtaaacagtaactgcgtgctataaggtggcactaaacttccttcttcccctttctatcggatttgtgcagccaggagggataccagtgacggctatagcgggagaaagacgggccctctcacaaccctttgccccttgcggttagtcatatagattttgcttgtgaagagaactggtcggttcaaagatgctttcttcaaagtactatagccttgcccactctctttctcccttctgatctcttgtatgctgattttactttaataagtgaaggtgatcaatcagtcaactaacaaattagccgccactgcaaggatattttctctgtcgttgttgttttcctacaaatataaacaaccgggtaacagtcaaagaaaaaaaaaaaaagaaaaggaaaaaaaaaaaatatgaaaaattttgttctgaaagaagcgatgagatgagtactcaatagtaacatataggcagcttacaccattaaacaaagaagcag

SEQ ID NO: 18 - DNA, 548 bp, Saccharomyces cerevisiae
ttaatcatcgtttactgccgcctatgagcgtaagctaatgttataaagaaacaagctataatattgttaaatatagttgatcaacagcattgtaatgattacaagagacgaggtggaatgaaccttatgaaatgcgttttatatataaactgtaataagagctaagttgaattgaaatctacgatacttgatgttgacattatagcactagttcccaggaaaccctttcgaaaaacacagcaaaaacaagagtactgtaaccaatgtaacatctgtacaccagggacccacacattaccaaaatcaaaattatttttctaatgcctgttatttttcctatttttcctctggcgcgtgaatagcccgcagagacgcaaacaattttcctcgcagtttttcgcttgtttaatgcgtattttcccagataggttcaaacctttcatctgtatcccgtatatttaagatggcgtttgctttctccgttgatttttttccttcttagtgatttttttgcattaaatcccagaacaatcatccaactaatcaag

SEQ ID NO: 19 - DNA, 352 bp, Saccharomyces cerevisiae
caacataaataatttctattaacaatgtaatttccataattttatattcctctccaccttctattgcatcatgtactattcaaatgactgtaacactagtattatgaagaaaacacccaaacatatctaggccatcagattttttttttttcatttttcatttttttctcattttcttatttatttttattgaaaaataataaccgacgcaaacaaattggaaaaaccaacgcaaaaaaaaaaagacgctaaattgtttataaaggcgaggaatttgtatctatcaattactattccagttgtcagtttacattgcttaccctctattatcacatcaaaacaactaattcga

SEQ ID NO: 20 - DNA, 548 bp, Saccharomyces cerevisiae
aatccaagtaaaaggatggatatcgttatactaaaagcaacacagaaaaggtccacgtcagttccacacaataacatttacgtagtgttcacgcgaagcagttacatctcaactaacataattgctggtgagcctacaacactgcatgcgtaaacgtcaacgggattacgttagtatttttggccgccggtaaattctcttgtttttttttcttgatttcacttcttttcatgttcctttggaataatctaattcctcatgattaaatgagactgttttttgtttccgtaacatccatacctttcctgtataatattcttgctgtaaagtttgttttttttatgaaaaaaacattttcttttcttgagatgaggcgccgcgagcctttctcccatgggcagtggtaaattttccaaatcaatgcagctctttgaaatacaacagcatttttcatacattttaagcaatttctagtttgtagatattgttagattagtttttgaacattgttttgataactgaaaataaaacagcaaacaaactacaaa

SEQ ID NO: 21 - DNA, 546 bp, Saccharomyces cerevisiae
cgtccaacgtgcgggtaccgtaccctgcagtgttgcaatcgtgtacttgccttatagtgtcagctattgatcaaggccacatgcaaaatagggaaggggggcattggcacaaaagagtggttagacgctcacaggggtgactacggttacaagtctaaatattttaagcccatcattaccggcaatgccctctgtacaggagttataagaaagattattcaatttcgcgcttgcattatgaaagaggttgcattcttcaatatcaggtgaaatgtgtcttgcctagacaatctaaaaaaggctgcacacccatgcatcattctaaaaaaattattttttttcttttcatttacttttcgttttttttttttttttcagttcgatttcttggtcggacgcgatggcaaatttttcatcgaggagattatcgttataaaggcctgttgattttcaaagagatagaaatcttctcttttaagtattctttttttaattaataaaaacacaagaaaactaatacagcaacagaaatacaaaagtatacaa

SEQ ID NO: 22 - DNA, 474 bp, Saccharomyces bayanus
cattcatctttcacctgccattagtaacccgacttctcattgagcgggttacggcagccacaggccacattccgaatgtctgggtgagcggtcccttttccagcatccactaaatatctcggatcccgctttttaatctggcttcctgaaaaaaatcaatggagtgatgcaaactgactggagcaaaaagctgacacaaggcaatcgacctacgtgtctgtctattttctcacaccttctattaccttctaactctctgggttggaaaaaactgaaaaaaaggttgtctccagtttccacaaatcatccccctgtttgattaataaatatataaagacgacaactatcgatcataaactcataaaactataactcctttacacttcttattttatagttattctattttaattcttattgattttaaaaccccaagaacttagtttcgaaaacacacacacacaaacaattaaa

SEQ ID NO: 23 - DNA, 410 bp, Saccharomyces mikatae
atgcttcaaaaacgcactgtactcctttttactcttccggattttctcgcactctccgcatcgccgcacgagccaagccacacccacacacctcataccatgtttcccctctttgtctctttcgtgcggctccattacccgcatgaaactgtataaaagtaacaaaagactatttcgtttctttttctttgtcggaaaaggcaaaaaaaaaaatttttatcacatttctttttcttgaaaattttttttgggattttttctctttcgatgacctcccattgatatttaagttaataaaagcactcccgttttccaagttttaatttgttcctcttgtttagtcattcttcttctcagcattggtcaattagaaagagagcatagcaaactgatctaagttttaattacaa

SEQ ID NO: 24 - DNA, 467 bp, Saccharomyces paradoxus
gttttatttctgctgccatccgtaaatgccaggatttgagcgggttacacaatatatctcatattttcggtgtctgggtcattactttactcttggcatccactaaatatattggatcctgctttttaaactggcttccagaaaaaaatcaatggagtgatgcaaactgcctggagtaaaagatgacacaaggcgattgacctacgcatgtatctatctcattttcttacaccttctatttcattctaactctttgatttggaaaacacctaagaaaaaaaaggttgaaatcagttccctgaaattgtccccctacttgactaataaatatataaagacggtaggtattgactgtaattcgtaaatctatacttcttaaacttcttcaaatttacttttttggatagtcttatttttggtttcaataccccaagaacttagtttcaaataaatacacatacaaacaa

SEQ ID NO: 25 - DNA, 414 bp, Saccharomyces paradoxus
atagccgacaaatcttttactcctttttttactcttccgcattttctcggactgcgcgcatcgccgcaccgcttccaaaacacctgaacattacatactatttttcccctctttctttctttagggtggtgttaaattacccgctctgaagctttggaaaagaaacaaaaggccacttcgtttctttttcttcgtcgaaaagggcaaaaaaaaaatttttaccacgtttctttttcttgaaaattttttttttttattttttctctttcgatgacctcccattgatatttaagttaataaatggtcatcaatttctcaagttttattttcgttttccttgtttcatgtcgacttttttacatccttctcagttagaaagaaagcatagcaatctaatctaagttttaattacaa

SEQ ID NO: 26 - DNA, 411 bp, Saccharomyces cerevisiae
atagcttcaaaatgtttctactccttttttactcttccagattttctcggacaccgcgcatcgccgtaccacttcaaaacacccaagcacagcatactaaatttcccctctttcttcctctagggtgtcgttaattacccgtactaaaggtttggaaaagaaaaaagtgaccgcctcgtttctttttcttcgtcgaaaaaggcaataaaaatttttatcacgtttctttttcttgaaaatttttttttttgatttttttctctttcgatgacctcccattgatatttaagttaataaacggtgttcaatttctcaagtttcagtttcatttttcttgttctattacaactttttttacttcttgctcattagaaagaaagcatagcaatctaatctaagttttaattacaa

SEQ ID NO: 27 - DNA, 550 bp, Saccharomyces cerevisiae
aaggcaagaaaaaaaattatgacccatacattttggtgcatgggtgatattttgtctttattattattagtaactttcagattaagacaaggaagaaagaaggaaaaaaggtaggacatgaagcatgagagataatcaaatttgctgaatgctgtgatactggtatacaaatagaagtgctgaagttcaagttatttggtaacatgcaagtttcattaatattttgttattatgtatttcaaggacaaaatgaattgtaggaagatgaaaaagtgatttcatcccccaacgattcatactctgggccgtacccttcttatcttacagacgcaaagtcagcagattcttgtattatggaactaagtaatttccgcgcacctattgttacgctgatatctgcgtacaaagtgacgtatttttaaaatatcttcgatatactgcaaaaacaaaagaacaattgacctcttaaccctttaaggtatggcataaagaacgctaagagctagcaaaagagtataccattcggatcgtgttgctaaagtcttgcgaa

SEQ ID NO: 28 - DNA, 285 bp, Saccharomyces cerevisiae
tttactgataatatacactctttggatcgagcccacttccagttggtaattggtgttccacaatttcagcattacatgtttttaaaccaaaattcggctccttttccctttttttcttattgggtggcgtgccgtacagaacgattggcttggtgtgaaatcaagagcaagcacaatagatatcaacatgaacaatatacaaaagtctctggcacagtttgactgcgttagaccaggctagggcatttctgaagctttacgtatcactagagaagttattttggc

SEQ ID NO: 29 - DNA, 264 bp, Saccharomyces cerevisiae
tggagtgaaggccagaattgttacttctgattttgtcgcatgtgttggtcaagccatgcctatggcaattttcattttttatttttcataattcctcattttggtgttcatggaggaagaggtccggcaagcgaatacacttccgtaagggatggcaagtacggccgttagcggtttaatacaacgtcagacagccttcatatgaatcacgtatatatgcttttatgattattctttcggcattttccttccttctcatactta

SEQ ID NO: 30 - DNA, 550 bp, Saccharomyces cerevisiae
ctggcgttttatcttttatgcatccaatatctaatattacttccgatcacgcatttagttctgattacagcagaaatcgtagcgcgatgagacatttcatcaaatggccttttttttttgggcaatttttttatatcttgaaatgatagttgccttgtactttcaaccgttcatttcattaagaacttgactaaatatgaacatttcttaaaaaaaaggttgacatataaaaataatcgaatataaacgatggaatttttataaaattaaacacatatatatatatatattaactataaatatgtcaaagaaaccatacaatcatagatttataactatcttttggatgacattaatgaacataacgctcctaatacaaatgtccaaaaaatattacccgcaaatacgaatcttttttttttctcgatgaaattttgcaaagagttcgaaatttttatttcaagagctggtagagaaaatttcataaggttttcctaccgatgcttttataaaatcttcgttttgtctcacatataccaacaagagtaac

SEQ ID NO: 31 - DNA, 492 bp, Kluyveromyces lactis
gttttatttaggttctatcgaggagaaaaagcgacaagaagagatagaccatggataaatgattatgttctaaacactcctcagaagctcatcgaactgtcatcctgcgtgaagattaaaatccaacttagaaatttcgagcttacggagacaatcatatgggagaagcaattggaagatagaaaaaaggtactcggtacataaatatatgtgattctgggtagaagatcggtctgcattggatggtggtaacgcatttttttacacacattacttgcctcgagcatcaaatggtggttattcgtggatctatatcacgtgatttgcttaagaattgtcgttcatggtgacacttttagctttgacatgattaagctcatctcaattgatgttatctaaagtcatttcaactatctaagatgtggttgtgattgggccattttgtgaaagccagtacgccagcgtcaatacactcccgtcaattagttgcac

SEQ ID NO: 32 - DNA, 654 bp, Saccharomyces cerevisiae
agtttatcattatcaatactcgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccagaacttagtttcgacgga

SEQ ID NO: 33 - DNA, 122 bp, Saccharomyces cerevisiae
caatattatttaaggacctattgttttttccaataggtggttagcaatcgtcttactttctaacttttcttaccttttacatttcagcaatatatatatatatttcaaggatataccattct

SEQ ID NO: 34 - DNA, 1024 bp, Kluyveromyces lactis
gctgtgaagatcccagcaaaggcttacaaagtgttatctcttttgagacttgttgagttgaacactggtgttttcatcaaacttaccaaggacgtgtacccattgttgaaacttgtatcaccatatattgttatcggacaaccttcacttgcatctatccgttctttaatccaaaagagatctagaataatgtggcaaaggccagaagataaagaaccaaaagagataatcttgaatgacaacaatatcgttgaagagaaattaggtgatgaaggtgtcatttgtatcgaggatatcatccatgagatttcgacgttgggcgaaaatttctcgaaatgtactttcttcctattaccattcaaattgaacagagaagtcagtggattcggtgccatctcccgtttgaataaactgaaaatgcgcgaacaaaacaacaagacacgtcaaatttcaaacgctgccacggctccagttatccaagtagatatcgacacaatgatttccaagttgaattgattaactataaaaggaaaatatctgtacaatagacatcgggctcccattggccctacccacatatgtagaaatacattactctattcactactgcatttagttatgtttaacatttgatatagcagactaccgccaggcacaatatattccccttccctcttgccattcgctgtacttgtggtggattccaattcagcgcagtcacgtgctagtaatcaccgcatttttttcttttcctttcaggctaaaaccggttccgggcctgatccctgcactcattttctaacggaaaaccttcagaagcataactacccattccagtttagagacatgacaggttcaacatcagatgcttcatatacttttatatattgaattatataaatatatctatgtactctaagtaagtacatctgctttaacgcattcctacatttgcttcgatttatttttattgttgatacctatttgaagaagtaaaaagtatcccacactacacagattatac

SEQ ID NO: 35 - DNA, 599 bp, Saccharomyces cerevisiae
caaacattaatttgttctgcatactttgaacctttcagaaaataaaaaacattacgcgcatacttaccctgctcgcgaagaagagtaacactaacgcattctatgggcaattgatgacagtattcagtacaagacatagtccgtttccttgattcaattcctatagcattatgaactagccgcctttaagagtgccaagctgttcaacaccgatcatttttgatgatttggcgtttttgttatattgatagatttcttttgaattttgtcattttcacttttccactcgcaacggaatccggtggcaaaaaagggaaaagcattgaaatgcaatctttaacagtattttaaacaagttgcgacacggtgtacaattacgataagaattgctacttcaaagtacacacagaaagttaacatgaatggaattcaagtggacatcaatcgtttgaaaaagggcgaagtcagtttaggtacctcaatgtatgtatataagaatttttcctcccactttattgtttctaaaagttcaatgaagtaaagtctcaattggccttattactaactaataggtatcttataatcacctaataaaatag

SEQ ID NO: 36 - DNA, 600 bp, Saccharomyces cerevisiae
ttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaa

SEQ ID NO: 37 - DNA, 600 bp, Saccharomyces cerevisiae
acatggcattaccaccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggcgacagccctccgacggaagtctctcctccgtgcgtcctcgtgttcaccggtcgcgttcctgaaacgcagatgtgcctcgcgccgcactgctccgaacaataaagattctacaatactagcttttatggttatgaagaggaaaaattggcagtaacctggccccacaaaccttcaaatcaacgaatcaaattaacaaccataggataataatgcgattagttttttagccttatttctggggtaattaatcagcgaagcgatgatttttgatctattaacagatatataaatgcaaaagctgcataaccactttaactaatactttcaacattttcggtttgtattacttcttattcaaatgtcataaaagtatcaacaaaaaattgttaatatacctctatactttaacgtcaaggagaaaaaactata

SEQ ID NO: 38 - DNA, 600 bp, Saccharomyces cerevisiae
taaggagatttcagattttttaatggaaagagaagttgtccaaaggagtataattattgacaaggatttggaatctgataatctgggtattactacggcaaacttcaacgatttctatgatgcattttataattagtaagccgatcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagttagaaaaagacatttttgctgtcagtcactgtcaagagattcttttgctggcatttcttctagaagcaaaaagagcgatgcgtcttttccgctgaaccgttccagcaaaaaagactaccaacgcaatatggattgtcagaatcatataaaagagaagcaaataactccttgtcttgtatcaattgcattataatatcttcttgttagtgcaatatcatatagaagtcatcgaaatagatattaagaaaaacaaactgtacaatcaatcaatcaatcatcacataaa

SEQ ID NO: 39 - DNA, 101 bp, Saccharomyces cerevisiae
cgaatttcttatgatttatgatttttattattaaataagttataaaaaaaataagtgtatacaaattttaaagtgacacttaggttttaaaacgaaaattc

SEQ ID NO: 40 - DNA, 106 bp, Saccharomyces cerevisiae
ataagaatataaagtaaacaattacgtaaccttagaaaaacagatataaaaaagttttacactgtttttaccacagtccatataaacttgtaattattacccgaat

SEQ ID NO: 41 - DNA, 120 bp, Saccharomyces cerevisiae
tacactaattttatgaaagctaacggaaaagagattagtgcttttggcttattacaaagtttgcggcaatattttccttatcagcatcataagctgtcagtatttcatgtattattagta

SEQ ID NO: 42 - DNA, 216 bp, Saccharomyces cerevisiae
caggccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaaca

SEQ ID NO: 43 - DNA, 172 bp, Saccharomyces cerevisiae
aataaaaaagaatatatactccacatgacatacgaaatatacgtatttattgttctgtatggaataacagcgattacataaagatgacatgttacttctttattcaaattaatcttgacgtgcaagggcctgcttgttatttcatcggacaatcccaacatcactttacacg

SEQ ID NO: 44 - DNA, 213 bp, Saccharomyces cerevisiae
acaagataataaaaaagataatattttcgtttaaaatttcagaaatattgcttacatcaaacgaaatagtaagcgtaaaccatatatcctttcaacgatatgtatcatttttatagtcttttggcggtataaaaagaatagccaaaggtggaagtcaggttaaaccaaaagaaactacccgcaagggatgtatgcatttgaacataaaaaact

SEQ ID NO: 45 - DNA, 75 bp, Saccharomyces cerevisiae
tttgatctgtagcctaagtataaaattctacgtatgtatatatttacatgcaattttttctttttccaattcatg

SEQ ID NO: 46 - DNA, 425 bp, Saccharomyces cerevisiae
cacttctcgattaacaaattcccagtattctttgaaatctatttttcttcctcaattgaatttgaataactgtctacgcgcactcctcctatctacaactacaacaaattttaaccactttattaccactttcctctttcatttatttttgtcttttatgttgtcaatttactagtatttttttttttttcatttacgttcaaggttttttatactcatttaacttgtcttaggttatttatatatatacctatatatttatatatatatatatatatgtatgtatatattattatcaccaaatgagaaataatagctaatttgatttttgattatttaaaatattggtttgttctttctgcaaacatctcgtttggtacgatattagtgaaaaacgatgtaattatcaacacgtgcattaccca

SEQ ID NO: 47 - DNA, 136 bp, Saccharomyces cerevisiae
ctgcaactcctcaatgtgtcaattaactcttacttaatttatgtatatattttttatgtatatgcttatatgcatgcgcatatgctcataaaagatacattgttataggtcatttcttttccaagctacatctagc

SEQ ID NO: 48 - DNA, 249 bp, Saccharomyces cerevisiae
cagatgacgggagacactagcacacaactttaccaggcaaggtatttgacgctagcatgtgtccaattcagtgtcatttatgattttttgtagtaggatataaatatatacagcgctccaaatagtgcggttgccccaaaaacaccacggaacctcatctgttctcgtactttgttgtgacaaagtagctcactgccttattatcacattttcattatgcaacgcttcggaaaatacgatgttgaaaat

SEQ ID NO: 49 - DNA, 287 bp, Saccharomyces cerevisiae
taggctaatatgaatgtatttgatctctattttattaatacgaaaccccttaataattgatattttcgatacatatttggcagtagttagctacgtaacagagtattattttcatttcaagttatgcatgaactctctaatttcacataccatgctaccactacccttggaggttttgttcatatcttttataataaagctaaaaccgaaaaggtgaagggaaaaaaaactattagagcctgtttcttgtatatagtaatatgtaatatttgcttcgtacgcttagt

SEQ ID NO: 50 - DNA, 149 bp, Saccharomyces cerevisiae
ctggttgatggaaaatataattttattgggcaaacttttgtttatctgatgtgttttatactattatctttttaattaatgattctatatacaaacctgtatattttttctttaaccaatttttttttttatagacctagagctgtact

SEQ ID NO: 51 - DNA, 228 bp, Saccharomyces cerevisiae
aagttttgttagaaaataaatcattttttaattgagcattcttattcctattttatttaaatagttttatgtattgttagctacatacaacagtttaaatcaaattttctttttcccaagtccaaaatggaggtttattttgatgacccgcatgcgattatgttttgaaagtataagactacatacatgtacatatatttaaacatgtaaacccgtccattatattgc

SEQ ID NO: 52 - DNA, 454 bp, Saccharomyces cerevisiae
cggattgagagcaaatcgttaagttcaggtcaagtaaaaattgatttcgaaaactaatttctcttatacaatcctttgattggaccgtcatcctttcgaatataagattttgttaagaatattttagacagagatctactttatatttaatatctagatattacataatttcctctctaataaaatatcattaataaaataaaaatgaagcgatttgattttgtgttgtcaacttagtttgccgctatgcctcttgggtaatgctattattgaatcgaagggctttattatattaccctttagcttattctgaggtttctgtggcgtgcaaagtgatgaaccgggcgggttttaaggataaaatcaaaaagtgaaaaaatgaacggaaaatggaatacctgtgaaatggagaatgataatgaatctttctgtcgtgcttgaaagattttcggct

SEQ ID NO: 53 - DNA, 216 bp, Saccharomyces cerevisiae
ttatgcatgtattgtacttgtattgccgtattattttttacagttaaaaaatgtgtacatataattatatagcgcccataatcaaatcagctcatacgtcaatttagtaataaaaaaaagcccttataaccttttagttaagaagattcaagattgcgatttgattaacgtcattacggaaatgtaaggacacaattaccaagagttacaaaatcg

SEQ ID NO: 54 - DNA, 239 bp, Ashbya gossypii
cagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcga

SEQ ID NO: 55 - DNA, 249 bp, Saccharomyces cerevisiae
agggaaccttttacaacaaatatttgaaaaattacctccattattataccttctctttatgtaattgttagttcgaaaattttttcttcattaatataatcaacttctaaaactttctaaaaacgttctctttttcgagattagtgcttcttcccaatccgtaagaaatgtttcctttcttgacaattggcaccagctggctactcgttgctcgaaaactactctcttttatttttaatttacgaacga

SEQ ID NO: 56 - DNA, 243 bp, Saccharomyces cerevisiae
cgctcaaaccaggcttttcttttccgtttttacgagctagataagcgcatccatatttactaatagatataatgagatatctgagatacatgtgtatgtatatatgcacgttttcttttattatctaaaaatcatattatattaagtaagagaaaaaaatgtacaactatataaatatatatttatttaaaatggttttgaatttttcctattctggttgatattgcccaaaagctattcagt

SEQ ID NO: 57 - DNA, 197 bp, Saccharomyces cerevisiae
aggacggttgctgaagaaaaaggctttttttattttgtccgttttttttttgtaaaacccaaagatctgaatctaaagcttttttaaacgtatatagatgtctacatgtgtgtttttgtttttttacgtacgtatacccacctatatatgcataatccgtaattgaaaaaaaaaaaagaaaaagatcaaggaacaca

SEQ ID NO: 58 - DNA, 241 bp, Saccharomyces cerevisiae
attctaaacgcatagttgtaaggttgatgtatatatatatatatatatgtatatattaattacaataatatgctcccgcccaaatttttctccttcaataccgccggaggcggtattgaaggaaatagacggagaattccttatcaagaaagcttccatcaaagtgtacataagaagtgccgaaattcgaagtattctttcagagagtatttttgcaacataccaataagccaaattactc

SEQ ID NO: 59 - DNA, 190 bp, Saccharomyces cerevisiae
ggaaaaaggtcttggctatatataacggcagacaaaatataagtatacacgtatatatggtggtaaacacgcatatactgtatgccatgtatttaccattacatagttatttacgcactctataaaaagttaacattgcattttaataaattccttaaattactctaattaggatggtagccctaccttt

SEQ ID NO: 60 - DNA, 117 bp, Kluyveromyces lactis
tatacaggaaacttaatagaacaaatcacatatttaatctaatagccacctgcattggcacggtgcaacactacttcaacttcatcttacaaaaagatcacgtgatctgttgtattg

SEQ ID NO: 61 - DNA, 137 bp, Kluyveromyces lactis
cagtcttttgtaacgaccccgtctccaccaacttggtatgcttgaaatctcaaggccattacacattcagttatgtgaacgaaaggtctttatttaacgtagcataaactaaataatacaggttccggttagcctgc

SEQ ID NO: 62 - DNA, 284 bp, Saccharomyces cerevisiae
gcggatctcttatgtctttacgatttatagttttcattatcaagcatgcctatattagtatatagcatctttagatgacagtgttcgaagtttcacgaataaaagataatattctactttttgctcccaccgcgtttgctagcacgagtaaacaccatccctcgcctgtgagttgtacccattcctctaaactgtagacatggtagcttcagcagtgttcgttatgtacggcatcctccaacaaacagtcggttatagtttgtcctgctcctctgaatcgtctc

SEQ ID NO: 63 - DNA, 295 bp, Saccharomyces cerevisiae
agcttttgattaagccttctagtccaaaaaacacgtttttttgtcatttatttcattttcttagaatagtttagtttattcattttatagtcacgaatgttttatgattctatatagggttgcaaacaagcatttttcattttatgttaaaacaatttcaggtttaccttttattctgcttgtggtgacgcgtgtatccgcccgctcttttggtcacccatgtatttaattgcataaataattcttaaaagtggagctagtctatttctatttacatacctctcatttctcattt

SEQ ID NO: 64 - DNA, 300 bp, Saccharomyces cerevisiae
ggagattgataagacttttctagttgcatatcttttatatttaaatcttatctattagttaattttttgtaatttatccttatatatagtctggttattctaaaatatcatttcagtatctaaaaattcccctcttttttcagttatatcttaacaggcgacagtccaaatgttgatttatcccagtccgattcatcagggttgtgaagcattttgtcaatggtcgaaatcacatcagtaatagtgcctcttacttgcctcatagaatttctttctcttaacgtcaccgtttggtctttt

SEQ ID NO: 65 - DNA, 400 bp, Saccharomyces cerevisiae
tctaatctctagtcattatttattcgcaaattcatttccctatacggcattcatacatatcattgttcacttcagtcctagcatatatcataaaatatacaattgttttctaattaccttacgttttttaaaagacttctataatacctcttttaactttacatgtagtcaaaataaagtgcagttccatcgatggtactttctcaccccggttgagtgatgttaacgatgtttaccgtataaaacttaattatattatatctttttttgcttatatgttatacatagaataaaaagttgattaaacacacattggtctgaaaacacgtgtagtactttctcctttgaagaataaaaaaagaaaataaagataataaaaacgaaaatagcgtacaattat

SEQ ID NO: 66 - DNA, 350 bp, Saccharomyces cerevisiae
ttggcattcttcaatttgatagacacttatccctgcatattttttttataaacagcttatagactttcatgtaaatttttcctaattaatgtattatttacttcgttaattttccgttgaattattgacatgttaaaggtgcactaaatatacctaatacaaaaaatggttttctgtggcaaatatatacagtggaaatttcagcatataatccctgctttactctttccttaagatttcgtcataattagaactttttttggtaaactgcattttctaccattattactttacatatgtatagctacaaaactgtattttgaagtgaaaagtatgatgaataaatgaat

SEQ ID NO: 67 - DNA, 225 bp, Saccharomyces cerevisiae
actagttttcttctttcctcctcttctttgaactgcttccaaattctgtctttaagtccatcacatggtgttttatgggattttgtattattacggtgttcggttttcttttgggtattgagcttttattttggtcttaatttttttttttctttttcaaaccatggactttattataattaatctacgacaacttttaatgattatttctttcttcaaatatac

SEQ ID NO: 68 - DNA, 716 bp, Artificial Sequence
  Synthetic Polynucleotide
aatggtttccaagggtgaagaattgatcaaggaaaacatgagaatgaaggttgtcatggaaggttctgtcaacggtcaccaattcaaatgtaccggtgaaggtgaaggtaacccatacatgggtactcaaaccatgagaatcaaggttattgaaggtggtccattaccatttgctttcgacatcttggctacttctttcatgtacggttccagaactttcatcaaatacccaaagggtattccagacttcttcaagcaatccttcccagaaggtttcacctgggaaagagttacccgttacgaggatggtggtgttgtcaccgtcatgcaagatacctctttggaagatggttgtttggtctaccacgttcaagtccgtggtgtcaacttcccatctaacggtcctgttatgcaaaagaaaaccaagggttgggaaccaaacactgaaatgatgtacccagctgacggtggtttgagaggttacactcacatggctttgaaggtcgatggtggtggtcacttgtcttgttctttcgtcaccacttacagatccaaaaagactgttggtaacatcaagatgccaggtattcatgccgttgaccacagattggaaagattggaagaatctgacaacgaaatgttcgttgtccaaagagaacacgctgttgccaaatttgctggtttgggtggtggtatggatgaattatacaagtaaa

SEQ ID NO: 69 - DNA, 698 bp, Artificial Sequence
  Synthetic Polynucleotide
aatgtccgaattgatcaaggaaaacatgcacatgaaattgtacatggaaggtactgttgacaaccaccacttcaagtgtacctctgaaggtgaaggtaagccttacgaaggtactcaaaccatgagaatcaaggttgttgaaggtggtccattaccatttgccttcgatatcttggctacctctttcttgtacggttccaagactttcatcaaccacactcaaggtattccagacttcttcaagcaatctttcccagaaggtttcacttgggaaagggttaccacctacgaagatggtggtgtcttgactgctacccaagacacttctctacaagatggttgtttgatttacaacgtcaagatcagaggtgtcaactttacctctaacggtccagtcatgcaaaagaaaactttgggttgggaagctttcaccgaaactttatacccagctgacggtggtttggaaggtagaaacgacatggctttgaaattggttggtggttctcatttgattgccaatgctaagaccacttacagatccaagaagccagccaagaacttgaagatgccagtctactacgttgactacagattggaaagaaaggaagctaacaacgaaacctacgttgaacaacacgaagttgctgttgctcgttactgtgatttgccatccaagttgggtcacaaattaaactaaa

SEQ ID NO: 70 - DNA, 719 bp, Artificial Sequence
  Synthetic Polynucleotide
aatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttaggttatggtttgatgtgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatcctatcaatctagattatccaaagatccaaacgaaaagagggatcacatggtcttgttagaatttgttactgctgctggtattacccatggtatggatgaattgtacaaataaa

SEQ ID NO: 71 - DNA, 719 bp, Artificial Sequence
  Synthetic Polynucleotide
aatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttaacttatggtgttcaatgtttttctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagggatcacatggtcttgttagaatttgttactgctgctggtattacccatggtatggatgaattgtacaaataaa

SEQ ID NO: 72 - DNA, 719 bp, Artificial Sequence
  Synthetic Polynucleotide
aatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttttcttatggtgttcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactttaactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccattcaatctgccttatccaaagatccaaacgaaaagagggatcacatggtcttgttagaatttgttactgctgctggtattacccatggtatggatgaattgtacaaataaa

SEQ ID NO: 73 - DNA, 719 bp, Artificial Sequence
  Synthetic Polynucleotide
aatgtctaaaggtgaagaattattcactggtgttgtcccaattttggttgaattagatggtgatgttaatggtcacaaattttctgtctccggtgaaggtgaaggtgatgctacttacggtaaattgaccttaaaattgatttgtactactggtaaattgccagttccatggccaaccttagtcactactttaggttatggtttgcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcactgctgacaaacaaaagaatggtatcaaagctaacttcaaaattagacacaacattgaagatggtggtgttcaattagctgaccattatcaacaaaatactccaattggtgatggtccagtcttgttaccagacaaccattacttatcctatcaatctgccttatccaaagatccaaacgaaaagagagatcacatggtcttgttagaatttgttactgctgctggtattacccatggtatggatgaattgtacaaataaa

SEQ ID NO: 74 - DNA, 713 bp, Artificial Sequence
  Synthetic Polynucleotide
aatggtaagtaagggtgaagaagacaatatggcgatcattaaggaattcatgcgtttcaaagtacacatggagggaagcgtgaacggacatgaatttgaaatcgaaggggaaggcgaaggtagaccatacgaaggaacccagaccgcaaagcttaaagttaccaaaggcgggccactaccatttgcatgggatatcttgagccctcagtttatgtatggcagtaaggcctacgttaaacacccagctgatattcccgactatttgaaattgtcttttccagaaggattcaaatgggaaagagtaatgaatttcgaggacggcggagttgttactgttactcaagattcaagtttgcaagacggtgaatttatttacaaggtcaaattaagagggactaatttccctagtgatggtcccgtcatgcaaaagaagactatgggttgggaagcctcatctgaacgtatgtatccagaagatggcgcgcttaagggggaaattaaacaaagattgaagttaaaagacggtggtcactacgacgcggaagttaagaccacttataaagctaaaaagcccgttcagttacctggtgcatataacgtaaacattaaattggatatcacttcacataatgaagattacactattgtggaacaatatgaaagagctgaaggtaggcactcaacgggtggaatggacgaattgtacaaataaa

SEQ ID NO: 75 - DNA, 806 bp, Artificial Sequence
  Synthetic Polynucleotide
aatgtccacaaaatcatataccagtagagctgagacacatgcaagtccggttgcatcgaaacttttacgtttaatggatgaaaagaaaaccaatttgtgtgcttctcttgacgttcgttcgactgatgagctattgaaacttgttgaaacgttgggtccatacatttgccttttgaaaacacacgttgatatcttggatgatttcagttatgagggtactgtcgttccattgaaagcattggcagagaaatacaagttcttgatatttgaggacagaaaattcgccgatatcggtaacacagtcaaattacaatatacatcgggcgtttaccgtatcgcagaatggtctgatatcaccaacgcccacggggttactggtgctggtattgttgctggcttgaaacaaggtgcgcaagaggtcaccaaagaaccaaggggattattgatgcttgctgaattgtcatccaagggttctctagcacacggtgaatatactaagggtaccgttgatattgcaaagagtgataaagatttcgttattgggttcattgctcagaacgatatgggaggaagagaagaagggtttgattggctaatcatgaccccaggtgtaggtttagacgacaaaggcgatgcattgggtcagcagtacagaaccgtcgacgaagttgtaagtggtggatcagatatcatcattgttggcagaggacttttcgccaagggtagagatcctaaggttgaaggtgaaagatacagaaatgctggatgggaagcgtaccaaaagagaatcagcgctccccattaaa

SEQ ID NO: 76 - DNA, 1091 bp, Artificial Sequence
  Synthetic Polynucleotide
aatgtctaagaatatcgttgtcctaccgggtgatcacgtcggtaaagaagttactgacgaagctattaaggtcttgaatgccattgctgaagtccgtccagaaattaagttcaatttccaacatcacttgatcgggggtgctgccatcgatgccactggcactcctttaccagatgaagctctagaagcctctaagaaagccgatgctgtcttactaggtgctgttggtggtccaaaatggggtacgggcgcagttagaccagaacaaggtctattgaagatcagaaaggaattgggtctatacgccaacttgaggccatgtaactttgcttctgattctttactagatctttctcctttgaagcctgaatatgcaaagggtaccgatttcgtcgtcgttagagaattggttggtggtatctactttggtgaaagaaaagaagatgaaggtgacggagttgcttgggattctgagaaatacagtgttcctgaagttcaaagaattacaagaatggctgctttcttggcattgcaacaaaacccaccattaccaatctggtcacttgacaaggctaacgtgcttgcctcttccagattgtggagaaagactgttgaagaaaccatcaagactgagttcccacaattaactgttcagcaccaattgatcgattctgctgctatgattttggttaaatcaccaactaagctaaacggtgttgttattaccaacaacatgtttggtgatattatctccgatgaagcctctgttattccaggttctttgggtttattaccttctgcatctctagcttccctacctgacactaacaaggcattcggtttgtacgaaccatgtcatggttctgccccagatttaccagcaaacaaggttaacccaattgctaccatcttatctgcagctatgatgttgaagttatccttggatttggttgaagaaggtagggctcttgaagaagctgttagaaatgtcttggatgcaggtgtcagaaccggtgaccttggtggttctaactctaccactgaggttggcgatgctatcgccaaggctgtcaaggaaatcttggcttaaa

SEQ ID NO: 77 - DNA, 656 bp, Artificial Sequence
  Synthetic Polynucleotide
aatgggtaggagggcttttgtagaaagaaatacgaacgaaacgaaaatcagcgttgccatcgctttggacaaagctcccttacctgaagaatcgaattttattgatgaacttataacttccaagcatgcaaaccaaaagggagaacaagtaatccaagtagacacgggaattggattcttggatcacatgtatcatgcactggctaaacatgcaggctggagcttacgactttactcaagaggtgatttaatcatcgatgatcatcacactgcagaagatactgctattgcacttggtattgcattcaagcaggctatgggtaactttgccggcgttaaaagatttggacatgcttattgtccacttgacgaagctctttctagaagcgtagttgacttgtcgggacggccctatgctgttatcgatttgggattaaagcgtgaaaaggttggggaattgtcctgtgaaatgatccctcacttactatattccttttcggtagcagctggaattactttgcatgttacctgcttatatggtagtaatgaccatcatcgtgctgaaagcgcttttaaatctctggctgttgccatgcgcgcggctactagtcttactggaagttctgaagtcccaagcacgaagggagtgttgtaaa

SEQ ID NO: 78 - DNA, 815 bp, Artificial Sequence
  Synthetic Polynucleotide
aatgacagtcaacactaagacctatagtgagagagcagaaactcatgcctcaccagtagcacaacgattatttcgattaatggaactgaagaaaaccaatttatgtgcatcaattgatgttgataccactaaggaattccttgaattaattgataaattgggtccttatgtatgcttaatcaagacacatattgatataatcaatgatttttcctatgaatccactattgaaccattattagaactttcacgtaaacatcaatttatgatttttgaagatagaaaatttgctgatattggtaataccgtgaagaaacaatatattggtggagtttataaaattagtagttgggcagatattactaatgctcatggtgtcactgggaatggagtagttgaaggattaaaacagggagctaaagaaaccaccaccaaccaagagccaagagggttattgatgttagctgaattatcatcagtgggatcattagcatatggagaatattctcaaaaaactgttgaaattgctaaatccgataaggaatttgttattggatttattgcccaacgtgatatgggtggacaagaagaaggatttgattggcttattatgacacctggagttggattagatgataaaggtgatggattaggacaacaatatagaactgttgatgaagttgttagcactggaactgatattatcattgttggtagaggattgtttggtaaaggaagagatccagatattgaaggtaaaaggtatagagatgctggttggaatgcttatttgaaaaagactggccaattataaa

SEQ ID NO: 79 - DNA, 383 bp, Artificial Sequence
  Synthetic Polynucleotide
aatggccgaccaagcgacgcccaacctgccatcacgagatttcgattccacggccgccttctatgaaaggttgggcttcggaatcgttttccgggacgccggctggatgatcctccagcgcggggatctcaagctggagttcttcgcccaccccgggctcgatcccctcgcgagttggttcagctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtcgacccggacgggacgctcctgcgcctgatacagaacgaattgcttgcaggcatctcataaa

SEQ ID NO: 80 - DNA, 1031 bp, Artificial Sequence
  Synthetic Polynucleotide
aatgggtaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggcaatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataaa

SEQ ID NO: 81 - DNA, 582 bp, Artificial Sequence
  Synthetic Polynucleotide
aatgggtaccactcttgacgacacggcttaccggtaccgcaccagtgtcccgggggacgccgaggccatcgaggcactggatgggtccttcaccaccgacaccgtattccgcgtcaccgccaccggggacggcttcaccctgcgggaggtgccggtggacccgcccctgaccaaggtgttccccgacgacgaatcggacgacgaatcggacgacggggaggacggcgacccggattcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctaaatgagacc

SEQ ID NO: 82 - DNA, 812 bp, Artificial Sequence
  Synthetic Polynucleotide
aatgggtaaggaaaagacacacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgtgtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaa

SEQ ID NO: 83 - DNA, 564 bp, Artificial Sequence
  Synthetic Polynucleotide
aatgggtagcccagaacgacgcccggtcgagatccgtcccgccaccgccgccgacatggcggcggtctgcgacatcgtcaatcactacatcgagacgagcacggtcaacttccgtacggagccgcagacaccgcaggagtggatcgacgacctggagcgcctccaggaccgctacccctggctcgtcgccgaggtggagggcgtcgtcgccggcatcgcctacgccggcccctggaaggcccgcaacgcctacgactggaccgtcgaatcgacggtgtacgtctcccaccggcaccagcggctcggactgggctccaccctctacacccacctgctgaagtccatggaggcccagggcttcaagagcgtggtcgccgtcatcggactgcccaacgacccgagcgtgcgcctgcacgaggcgctcggatacaccgcgcgcgggacgctgcgggcagccggctacaagcacgggggctggcacgacgtggggttctggcagcgcgacttcgagctgccggccccgccccgccccgtccggcccgtcacacagatctaaatgagacc

SEQ ID NO: 84 - DNA, 677 bp, Artificial Sequence
  Synthetic Polynucleotide
aatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgaatcgtggcaagaataccaagagttcctcggtttgccagttattaaaagactggtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagaatctaacaaaatagcaaatttcgtcaaaaatgctaagaaataaa

SEQ ID NO: 85 - DNA, 2643 bp, Artificial Sequence
  Synthetic Polynucleotide
ttaccaatgcttaatcagtgaggcacctatctcagcgatctgtctatttcgttcatccatagttgcctggctccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgggtctcagtgcaggtcttctgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagaatcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtagggaaacctgtcgtgccagctgcattaatgaatcggccaacgcgcggggaagacctaatgagagaccgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagcccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacag

SEQ ID NO: 86 - DNA, 2643 bp, Artificial Sequence
  Synthetic Polynucleotide
gacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgggtctcaaatgaggtcttctgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagaatcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtagggaaacctgtcgtgccagctgcattaatgaatcggccaacgcgcggggaagaccttaaaagagaccgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagcccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctggctccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacct

SEQ ID NO: 87 - DNA, 2643 bp, Artificial Sequence
  Synthetic Polynucleotide
gacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgggtctcataaaaggtcttctgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagaatcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtagggaaacctgtcgtgccagctgcattaatgaatcggccaacgcgcggggaagacctcctcagagaccgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagcccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctggctccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacct

SEQ ID NO: 88 - DNA, 4483 bp, Artificial Sequence
  Synthetic Polynucleotide
agtgagttgattggaagacctgacatattcttaccaatcctttcataagctaattatgccatccatatagcaagagaatccggtgggggcgccatgcctatccggcggcaacattattactctggtatacgggcgtaactccataatatgccaccacttacctttaacatgttcatggtaggtaccccacccagccataaggaaattttcaaaggcgttggatcaaaaaataggcctttatttcatcgcgtgattgaggagcataacatgtttagtgaaggtttcttttggaaaacttcagtcgctcattattagaaccagggaggtccaggctttgctggtgggagagaaagcttatgaagctggggttgcagatttgtcgattggtcgccagtacacagttttaaaaagtcagagaatgtagagaagtatggatctttgaaaccctaagcgacttccaatcgctttgcatatccagtaccacacccacaggcgtttgtgcagagacctgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagaatcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcgggggtctcacctcttgcccatcgaacgtacaagtactcctctgttctctccttcctttgctttcttcgtacgctgcaggtcgacgaattctaccgttcgtataatgtatgctatacgaagttatagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcataacttcgtataatgtatgctatacgaacggtagaattcgatatcagatccactagtggcctatcggatcgatgtacacaaccgactgcacccaaacgaacacaaatcttagcaaggtcttcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagcccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagagttctgaggtcattactggatctatcaacagcagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaactcgcgcgtttgccacctgacgtctaagaaaaggaatattcagcaatttgcccgtgccgaagaaaggcccacccgtgaaggtgagcc

SEQ ID NO: 89 - DNA, 3027 bp, Artificial Sequence
  Synthetic Polynucleotide
gttgattggaagacctgtgcagcttgccttgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaacaatgggtaccactcttgacgacacggcttaccggtaccgcaccagtgtcccgggggacgccgaggccatcgaggcactggatgggtccttcaccaccgacaccgtattccgcgtcaccgccaccggggacggcttcaccctgcgggaggtgccggtggacccgcccctgaccaaggtgttccccgacgacgaatcggacgacgaatcggacgacggggaggacggcgacccggattcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctaaacagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgacctcaggtcttcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagcccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagagttctgaggtcattactggatctatcaacagcagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaactcgcgcgtttgccacctgacgtctaagaaaaggaatattcagcaatttgcccgtgccgaagaaaggcccacccgtgaaggtgagccagtga

SEQ ID NO: 90 - DNA, 30 bp, Artificial Sequence
  Synthetic Polynucleotide
ttaccaatcctttcataagctaattatgcc

SEQ ID NO: 91 - DNA, 34 bp, Artificial Sequence
  Synthetic Polynucleotide
catcttcaatgttgtgtctaattttgaagttagc

SEQ ID NO: 92 - DNA, 22 bp, Artificial Sequence
  Synthetic Polynucleotide
gtgcggccatcaaaatgtatgg

SEQ ID NO: 93 - DNA, 39 bp, Artificial Sequence
  Synthetic Polynucleotide
ttatgttcaagaaagaactatttttttcaaagatgacgg

SEQ ID NO: 94 - DNA, 23 bp, Artificial Sequence
  Synthetic Polynucleotide
taccctccttgacagtcttgacg

SEQ ID NO: 95 - DNA, 60 bp, Artificial Sequence
  Synthetic Polynucleotide
catagtgtcgggaacaggtcattctaaaaaaagtaaaataaaattggatggcggcgttag

SEQ ID NO: 96 - DNA, 60 bp, Artificial Sequence
  Synthetic Polynucleotide
cgattcgatactaacgccgccatccaattttattttactttttttagaatgacctgttcc

SEQ ID NO: 97 - DNA, 15 bp, Artificial Sequence
  Synthetic Polynucleotide
ttgtgaccgccctgc

SEQ ID NO: 98 - DNA, 449 bp, Artificial Sequence
  Synthetic Polynucleotide
catcttgtagttatgactgagccaattgattaaacccacaaggatatacaataatgagaggaaattcagaacatttaattttttttctcgtcggcacgcgggttcagcaatgttgagcttcatctctataaatcgcatttttggataaatgttcacaaaataatatcgaggataccctttaaatataaaccacacggttccattttatatcttagctatacatctttcctgatgattcatcagctatcgcacgcagtaaccggcttcagtcgaaattgttttcttgcggatacgtattcgacccagagattttagtccattattttattgagacactctactccggttcgggtatttggtaaacttagttattcgcgctctccgctcaacaattaaaaaaatcctattatgtttattgagcttcagcaatatttaaaaatacttgcaca

SEQ ID NO: 99 - DNA, 449 bp, Artificial Sequence
  Synthetic Polynucleotide
atttggccgtttacaccttgagttaatcaatacgtatttaagtacacaagcgttactttaactaccgctccttccttttgcatagcgtacagtatttgtttatatatgttcaccggtggtacaaagtttaaatttttattatatgcctctcaatttcttaattagagatgatatgaattcacacattgattaattcataaagtgacttctcttgagagataacaattaacgcataccttatcatgcacttacgcaaacgcatgtctctaacctaacaaactttgcgctacaagtttcggctttgtttatagtgaaatggcagagcggtaggaacaccattcttactttgctcctgatcctgcgtactatattctaacaatatgataatatttgatacaaactctggaaagagcggcttcgagtgatgagaaaccctaagctctccattg

SEQ ID NO: 100 - DNA, 450 bp, Artificial Sequence
  Synthetic Polynucleotide
tttttttttttttttttttattgatgttaccctgaaaaaacccagacacgctcaatatttctctgtcacccggcctttttttttttttttgaaagggtttagtaccacatgctatgatgcccactgtgatctccagagcaaagttcgttcgatcgtactgttactctctctctttttttcaaacagaattgtccgaatcgtgtgacttcaatagcctgttctcacacactcttttcttctaaccttgtttgtggtttagtttagtagaacctcgtgaaacttacatttacatattgatttttttctctttctgtagtatataagtatttttttttatagtatataatgtagtatcttcttctgttctttttttcttagttcttttctttctatagttcttatctttgttcttttatactttcttttataattaaacaattaaaaacaaaa

SEQ ID NO: 101 - DNA, 458 bp, Artificial Sequence
  Synthetic Polynucleotide
aatagattggaaatactgtgtatcgacacctggacatctcgtttgtgtggacttcgactgtttcatagccttcagtcacccgttgtttgcaatgcccaagattattccaaacttaagctagacaaattggttattcccctatgctgttttttgctattcaaatcagaattattatatattcacccgtcggtgtgtggcatctctagcgaaagtactaaaattattatttacacccaagcataaggacgcattatcgcatgactgtgaaataaaattttacgacttcctagttgcaatcagaaaatacgtccagttataaataataatgctactaagcctgataaatatagtcctcttgactaatattgttccgtattcgtattttttctattttccagacttttcaatgacctaaacattacggattaagcatatgactaaaaaacaaaaaaaacaaa

SEQ ID NO: 102 - DNA, 458 bp, Artificial Sequence
  Synthetic Polynucleotide
atgtaccacaaaaagattcaattgttatctcccgtaataaagacacactgcctggtaagccttaaattgcgtcatcggcatccatggctatatgtattcgggtacgctaactttcaattcgtttcttacccgtccctgtggtgagggtgtttgccaaaattcactcagctcatcttccgcgacttcctttagatcaatcatgcattgagcaacactttataattttttacgacttcctcttaagattcaaacctcgcagacaccaatttttttttttttcgacttccttggggtatttgggggttacgcaattattgtaatcggttattgagaatgacaccacaagtcattggccttttcgtatctataattactatcatatattttccatacattgttcttctcaaacgaagcaaataatttaactcgctagattcgagaaaaacaaaaaaaacaaa

SEQ ID NO: 103 - DNA, 458 bp, Artificial Sequence
  Synthetic Polynucleotide
ctctctccctgctcccggcttagtatatagaaaaggttaatattctttaagaaatggcacaaaacttacgacaagcagcttcctgcccaaattatttcttaagcctagctcctgctgagatttgaacacctggacatattatatttttaattgcgcactatttaatattagatgtatatttcttaactgtagaatcggccatgtgtcgaatattcctttttttttttttcgacttcctatgctatcggattttaaagtctgtcggtttttaataattttacacctggacattatcattctatgctacgcaactcttaagcttgaattgttttgggatagatagcaaaatttcttaccacaagctcatgtttccattagttttgactaatgaattaacttgttttcaaataaataaaattaaacctacaaaagcgagcactatggaaggatctccgtat

SEQ ID NO: 104 - DNA, 458 bp, Artificial Sequence
  Synthetic Polynucleotide
agtatcaccacgacgaccagtgttttagatgatacattgcttcaatactcgagggggctccccttttgttgaacatcacccgtgtttgatatatacgctattataatctgaaattattcaaatggttacccgttttacttaactgacctcttgcttgaaagtagtcacaacgtaagtctggctttgattcaaatttgtacgagctctacaattccttaattatttaaatagccgtaaatagttatcttccaagatatcctccactgaaaaaaaaaaaaaagttcgatatcctgactagctgaattacgtgttcagatattaatgatccgattcacgacaattagcattactggattatgcatttctagtcagaaattactgatatagttgttctcttataaaactgcacactgagcgtactaaaatcagtaggtaaccagaaaaataacaaaagtaaa

SEQ ID NO: 105 - DNA, 458 bp, Artificial Sequence
  Synthetic Polynucleotide
actagtttaggtggcaatttcttgatggcaaaaaatcacaagtcgactggcctcgttacagacttcaatacttgtgttgattaagatataggctgaaggtcctaatcggatcccttaatttcaagtttattaatgcccaccttttctctattgcagctggcccttggttcaagaaaatcgaagcgtctgaacggtggttataaagatgtttttgtaaacttcttaataacttgaatcgtaaattatcgacatttctcctacctaacttttttttttttttacccagatcctgcgcgttgtcttttacgaaaaccattagattactcgcaaaggggtatagttatgctgattacacttagtaatgtccattataaagatagtatatctctccaagcgcaattggctagaactctgacttataacacagcatgagacaaatggtcttttgcgtagtaaaa

SEQ ID NO: 106 - DNA, 458 bp, Artificial Sequence
  Synthetic Polynucleotide
aaatatccgtcaagagcatttaaaggaggcgttcagttagttcccctagtctggatacagtcattatttttcttaactactgtttgtgtatgtactccacaggacactaaagatatctgtattgatattagcgcgaattccgtcctaactttgccatgtttggtttgcatgacactatatacgatcccatattggcgacagatatatattttgataacaccgtatactggacaatgcccactatacgatcttcgagtaacaacgtctgctgatctaacacagcttccttaatgagcatttgtcttacgtatgaatcagctcgaacaaaatatccatatgtagtatggatgccaatttggcattattaattcaacgcaaaggtactgacttctgataacggttctcaccgttactctccatcatcataatgctgagagtttagaaatagaacagaatgc

SEQ ID NO: 107 - DNA, 458 bp, Artificial Sequence
  Synthetic Polynucleotide
cttctaaacgcagccgaacgccaggactattaaggtttcattcttgattcttatgtatatttttgggctcgtgcggaaattgatgaatgaatgcgtttttgtcactccttaacctaccatatcgataaagaatccctgttaaaacatatgttgcttatggtatactctcagatcacgtgtttgtgggcacgggaaataattttgcaagcactaattgaataaatctgatatatgacaacttgaactttagttggagctaagggccttctttaacctcttctctttgctcaacctacaatctcagtacgggattaggaattctggaataaatgtaccttacgataacccatatgtatcctaatgcgtcaagagacgatatatgttcacaatatacctctgaagcgcaccgtcatcgttcaaatcgaagtgcactttgataagtcattatccaagatagt

SEQ ID NO: 108 - DNA, 458 bp, Artificial Sequence
  Synthetic Polynucleotide
ggtccacacttattactgacttttctacatctatataagccatgcgagataattgtttctatccttatcaacacaatgagttttaacgcatgcttaagctctagtggttacacgtatgggtgtacaagaatcactgcaggcgttagtattttgcgttaatgagtagataactagtgaatttcttcgttatactactataattcgggcataagttttgtactctattggcataggatccgatacggacctgcccgagcaataccgtcataaacttttgtacagcccttttcaagtcacgtcactttacgtatattaatcgaattaaagtcgtcgtatactagggccacgaatatgtacctgtaacatacggatattaagctctcacgatcaaagtaaagatagcgcaatcgtaagatgaatctgattgccttgatctctctttaatcactccaccagtg

SEQ ID NO: 109 - DNA, 13 bp, Artificial Sequence
  Synthetic Polynucleotide
ttttttttttttt

SEQ ID NO: 110 - DNA, 13 bp, Artificial Sequence
  Synthetic Polynucleotide
ttaatttaatttt

SEQ ID NO: 111 - DNA, 12 bp, Artificial Sequence
  Synthetic Polynucleotide
acacccaagcat

SEQ ID NO: 112 - DNA, 13 bp, Artificial Sequence
  Synthetic Polynucleotide
accccttttttac

SEQ ID NO: 113 - DNA, 13 bp, Artificial Sequence
  Synthetic Polynucleotide
atcatctatcacg

SEQ ID NO: 114 - DNA, 13 bp, Artificial Sequence
  Synthetic Polynucleotide
gtcattttacacg

SEQ ID NO: 115 - DNA, 17 bp, Artificial Sequence
  Synthetic Polynucleotide
tttccgaaaacggaaat

SEQ ID NO: 116 - DNA, 17 bp, Artificial Sequence
  Synthetic Polynucleotide
ataccaaatacggtaat